(19) United States
(12) Patent Application Publication
(10) Pub. No.: US 2021/0310057 A1
(43) Pub. Date: Oct. 7, 2021

(54) KINETICALLY PROGRAMMED SYSTEMS AND REACTIONS FOR MOLECULAR DETECTION

(71) Applicant: VALORISATION-RECHERCHE, LIMITED PARTNERSHIP, Montreal (CA)

(72) Inventors: Alexis VALLEE-BELISLE, Outremont (CA); Guichi ZHU, Montreal (CA)

(21) Appl. No.: 17/057,202

(22) PCT Filed: May 31, 2019
(86) PCT No.: PCT/CA2019/050756

§ 371 Date: Nov. 20, 2020

Related U.S. Application Data
(60) Provisional application No. 62/680,784, filed on Jun. 5, 2018.

(51) Int. Cl. C12Q 1/6837

(52) U.S. Cl. CPC C12Q 1/6837

(57) ABSTRACT

The present disclosure concerns detection systems which are kinetically controlled. In the systems of the present disclosure, the systems are kinetically controlled by a first association constant (k₁) between the targeting oligonucleotide and the target, a second association constant (k₂) between the targeting oligonucleotide and the signaling oligonucleotide and a third association constant (k₃) between the signaling oligonucleotide and an anchoring nucleotide. The system is configured such that k₁>k₂>k₃.

### CROSS-REFERENCE TO RELATED APPLICATIONS

This present application claims priority from U.S. provisional application 62/680,784 filed on Jun. 5, 2018 and herewith incorporated in its entirety.

### TECHNOLOGICAL FIELD

The present disclosure concerns the use of oligonucleotides which are kinetically programmed to detect and optionally quantify, in a kinetically-controlled fashion, a target in a sample suspected of containing same.

### BACKGROUND

While living organisms have developed various innovative chemical systems to detect thousands of different molecules in seconds directly in complex biological sample (e.g. binding-induced biomolecular switches, enzymes, riboswitches . . . ), current assays for the quantitative detection of molecules still mostly rely on complex, multi-steps, wash- and reagent-intensive chemistry (e.g., enzyme-linked immunosorbent assays (ELISA), Western blots, HPLC, and polarization assays) that necessitate specialized technicians and require several hours before completion. The development of nature-inspired “one-pot” reactions for the quantitative detection of multiple molecules would drastically simplify molecular detection methods and would permit applications ranging from diagnostic to disease treatment and monitoring.

In the last 20 years, various nature-inspired strategies have been exploited to develop easy-to-use “one-step” biosensors. Among these, a popular strategy typically consists in engineering bio-recognition elements in such a way that these can signal a specific binding events using signaling mechanisms such as electrochemistry, fluorescence, SPR, colorimetric and Raman. Although these strategies have seen some success, they typically suffer from three main limitations. First, they are hardly generalizable: each new recognition elements-target molecule pair is structurally distinct and must be re-engineer to provide a high gain signaling mechanism (see for example aptamer-based sensors—E-AB sensors). Second, those recognition-based sensors typically fail in whole blood because their signaling mechanisms are generally too sensitive to non-specific adsorption of blood protein on the sensor surface. Three, several types of sensing mechanisms require long equilibration time, making those mechanisms too slow (>10 min) for point-of-care applications.

It would be highly desirable to be provided with kinetically programmed systems and reactions for molecular detection of one or more targets in complex mixture, such as whole blood.

### BRIEF SUMMARY

One aim of the present invention is to provide a system comprising kinetically programmed elements for the kinetically controlled detection of one or more targets.

According to a first aspect, the present disclosure provides a system for detecting a target in sample. The system comprises a plurality of anchoring oligonucleotides; a first substrate having a surface associated with, at a plurality of discrete locations, each of the first end of the plurality of anchoring oligonucleotides; a plurality of signaling oligonucleotides and a plurality of targeting oligonucleotides. Each of the plurality of anchoring oligonucleotides has a first end and a second free end. Each of the plurality of signaling oligonucleotides has a first nucleic acid sequence which is substantially complementary to a first region of each of the anchoring oligonucleotides and is capable of hybridizing with the anchoring oligonucleotide; has a second nucleic acid sequence which is substantially complementary to a second region of each of a plurality of targeting oligonucleotides and is capable of hybridizing with the targeting oligonucleotide, wherein the first nucleic acid sequence and the second nucleic acid sequence are configured to avoid simultaneous hybridization of the signaling oligonucleotide with the anchoring oligonucleotide and the targeting oligonucleotide; has a first end being associated with a reporter moiety and a second free end; and is configured such that, upon hybridizing with the anchoring oligonucleotide, the first end of the signaling oligonucleotide is located in the vicinity of the first end of the anchoring oligonucleotide. Each of the plurality of targeting oligonucleotides is capable of specifically binding to the target; has the second region which is substantially complementary to the second nucleic acid sequence of each of the signaling oligonucleotides and is capable of hybridizing with the signaling oligonucleotide and has a dissociation constant (K_{D}) with the target lower than the concentration of the target in the sample. The system is (i) kinetically controlled by a first association constant (k₁) between the targeting oligonucleotide and the target, a second association constant (k₂) between the targeting oligonucleotide and the signaling oligonucleotide and a third association constant (k₃) between the signaling oligonucleotide and an anchoring nucleotide and (ii) configured such that k₁>k₂>k₃. In an embodiment, the system is configured such that k₁ is at least 10 times higher than k₂. In an embodiment, the system is configured such that k₂ is at least 10 times higher than k₃. In still another embodiment, the molar concentration of the plurality of targeting oligonucleotides is equal to or higher than the molar concentration of the plurality of signaling oligonucleotides.

In a further embodiment, the molar concentration of the plurality of the signaling oligonucleotides is equal to or higher than the concentration of the plurality of anchoring oligonucleotides associated with the surface of the first substrate. In still another embodiment, the targeting oligonucleotide and/or the signaling oligonucleotide is configured so as to avoid hybridization between to the targeting oligonucleotide and to the signaling oligonucleotide in the presence of the target. In another embodiment, the first end of each of the anchoring oligonucleotides is covalently associated to the surface of the first substrate. In still a further embodiment, each of the anchoring oligonucleotide comprises at least 8 nucleic acid bases. In yet another embodiment, the first region of the anchoring oligonucleotide is substantially identical to the second region of the targeting oligonucleotide. In still a further embodiment, the first region of the anchoring oligonucleotide is complementary over the entire length of the first nucleic acid sequence of the signaling oligonucleotide. In another embodiment, the first substrate is a metallic electrode, such as, for example, a gold electrode. In still a further embodiment, the first nucleic acid sequence of the signaling oligonucleotide is complementary over the entire length of the first region of the anchoring oligonucleotide. In yet another embodiment, the second nucleic acid sequence of the signaling oligonucleotide is complementary over the entire length of the second region of the targeting oligonucleotide. In another embodiment, the first nucleic acid sequence is the second nucleic acid sequence. In yet another embodiment, each of the signaling oligonucleotide comprises at least 8 nucleic acid bases. In still another embodiment, the reporter moiety is a redox-reporter, such as, for example, methylene blue. In yet a further embodiment, the targeting oligonucleotide is an aptamer. In an embodiment, the second region of each of the targeting oligonucleotides is substantially identical to the first region of the anchoring oligonucleotide. In yet another embodiment, the second region of each of the targeting oligonucleotide is complementary over the entire length to the second nucleic acid sequence of the signaling oligonucleotide. In an embodiment, the targeting oligonucleotide is at least 10 nucleic acid bases. In another embodiment, the system is for detecting a plurality of distinct targets. The multiplex system comprises a plurality of types of anchoring oligonucleotides each anchoring oligonucleotide type having a distinct nucleic acid sequence, a first end and a second free end. The multiplex system also includes a plurality of types of substrates, each of the substrate type having a surface associated with, at a plurality of discrete locations, with the first end of a single type of anchoring oligonucleotides and each of the substrates having a different type of anchoring oligonucleotide. The multiplex system further comprises a plurality of types of signaling oligonucleotides, wherein each type of the signaling oligonucleotides: has a first nucleic acid sequence which is substantially complementary to a first region of a corresponding anchoring oligonucleotide type and is capable of hybridizing with the corresponding anchoring oligonucleotide type; has a second nucleic acid sequence which is substantially complementary to a second region of each of a plurality of targeting oligonucleotide type and is capable of hybridizing with the targeting oligonucleotide; wherein the first nucleic acid sequence and the second nucleic acid sequence are configured to avoid the simultaneous hybridization of the signaling oligonucleotide with the anchoring oligonucleotide and the targeting oligonucleotide; has a first end being associated with a reporter moiety and is configured such that, upon hybridizing with the corresponding anchoring oligonucleotide, the second end of the signaling oligonucleotide is located in the vicinity of the first end of the anchoring oligonucleotide. The multiplex system further includes a plurality of types of targeting oligonucleotides wherein each type targeting oligonucleotides: is capable of specifically binding to a corresponding target; has the second region which is substantially complementary to the second nucleic acid sequence of each of the signaling oligonucleotides and is capable of hybridizing with the signaling oligonucleotide and has a dissociation constant (K_{D}) with the target lower than the concentration of the target in the sample.

According to a second aspect, the present disclosure provides a method for the detection of a target in a sample. Broadly, the method comprises (a) providing the sample suspected of having the target and the system described herein (which comprises a plurality of anchoring oligonucleotides, a first substrate, a plurality of signaling oligonucleotides and a plurality of targeting oligonucleotides); (b) providing or determining a control amount of the plurality of anchoring oligonucleotide having hybridized with the signaling oligonucleotide in the absence of the target; (c) contacting the sample with the plurality of signaling oligonucleotides and the plurality of targeting oligonucleotides in the absence of the anchoring oligonucleotides to provide a targeted mixture; (d) contacting the targeted mixture with the plurality of anchoring oligonucleotides associated with the first substrate to provide a detectable mixture; (e) determining a test amount of the plurality of anchoring oligonucleotides having hybridized with the plurality signaling oligonucleotides in the system in the presence of the detectable mixture; and (f) characterizing the sample has having the target if it is determined that the test amount is higher than the control amount and as lacking the target if it is determined that the test amount is equal to or lower than the control amount. In the present disclosure, the method is (i) kinetically controlled by a first association constant (k₁) between the targeting oligonucleotide and the target, a second association constant (k₂) between the targeting oligonucleotide and the signaling oligonucleotide and a third association constant (k₃) between the signaling oligonucleotide and an anchoring nucleotide and (ii) configured such that k₁>k₂>k₃. In an embodiment, the method further comprises quantifying the concentration of the target in the sample based on the test amount. In still another embodiment, the method further comprises determining a gain between the test amount and the control amount. In an embodiment, the method comprises determining or quantifying the test amount and/or the control amount electrochemically. In a further embodiment, the method is for detecting a plurality of distinct targets and comprises providing the multiplex system described herein. In a further embodiment, the method comprises incubating the sample with the plurality of targeting oligonucleotides prior to contacting the sample with the plurality of signaling oligonucleotides. In a further embodiment, the method further comprises determining a dissociation constant (K_{D}) between the target and the targeting oligonucleotide. In still another embodiment, the method further comprises including a source of cations prior to or at step (c); including a source of cations prior to or at step (d) and/or applying a voltage to the substrate prior to or at step (d). In an embodiment, the sample is a biological sample, such as, for example, whole blood. In yet another embodiment, the sample is a food sample.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 provides a schematic representation of an embodiment of a kinetically programmed one-pot reactions for molecular detection. The «one-pot» three reactions system enables to quantitatively detect the concentration of ligand L through specific binding to receptor R. Receptor R can competitively bind either L or S, a signaling molecule. In absence of ligand (L), the receptor (R) is available to sequester S, the signaling molecule (formation of the R-S complex), therefore preventing S from interacting with the anchoring molecule D. In presence of L, this latter binds to the receptor (formation of the L-R complex) preventing this later from binding to the signaling molecule S. S is now free to bind to the anchoring molecule, D, thus generating an output signal. Simulations show that optimal efficiency and gain of this one-pot sensing mechanism will be reached before equilibrium when k₁>k₂>k₃ (see FIG. 2).

FIGS. 2A to 2E provide a simulation of the kinetically programmed one-pot reaction and it reveals that high gain response can be obtained rapidly when k₁>k₂>k₃. In this simulation, the DG of binding of L-R, R-S and S-D were set to similar value and were kept constant even when changing kₒₙ. (FIG. 2A) Simulation assuming that k₁=k₂=k₃. (FIG. 2b) Stimulation assuming that k₁=10k₂=100k₃. (FIG. 2C) Simulation assuming that k₁=100k₂=10 000k₃. (FIG. 2D-E) Simulations demonstrate that optimal efficiency and gain of this one-step sensing mechanism is reached before equilibrium when k₁>k₂>k₃.

FIGS. 3A to 3E illustrate (FIG. 3A) a kinetically programmed, one-pot reaction for the detection of quinine. The figure shows the receptor aptamer (SEQ ID NO: 1), the signaling DNA (SEQ ID NO: 4) and the anchoring DNA (SEQ ID NO: 8) (FIG. 3B) Electrochemical (square wave voltammetry) currents produced following the Signaling-Anchoring complex formation in absence or presence (100 μM quinine) of quinine after 5 mins. (FIG. 3C) Kinetic of Signaling-Anchoring complex formation versus time with (+) and without (−) quinine (k_{obs}^{+quin}=0.058 nM⁻¹ k_{obs}^{-quin}=0.085 nM⁻¹ min⁻¹). (FIG. 3D) Difference of electrochemical signal (signal gain) obtained in presence of 100 μM quinine versus time. (FIG. 3E) Difference of electrochemical signal (signal gain) obtained after 30 minutes versus quinine concentration (C_{50%}=32.1±8.6 μM). The errors bars show the standard deviation of current obtained from three electrodes.

FIGS. 4A to 4C illustrate that the one-pot assay is kinetically controlled (does not reach equilibrium) and requires careful optimization of its reaction rates (k₁>k₂>k₃). Performing the one-pot assay by: (FIG. 4A) pre-equilibrating the aptamer and quinine (target) for 30 mins; (FIG. 4B) pre-equilibrating the aptamer and signaling strand for 30 mins; (FIG. 4C) pre-equilibrating the signaling and anchoring DNA for 30 mins.;

FIGS. 5A and 5B illustrate that optimizing the gain of this specific one-pot assay can be achieved by increasing k₂ (i.e. increases the difference between k₂ and the slow k₃). Here, k₂ was increased by increasing the concentration of the aptamer from 50 nM to 200 nM (reactions rates were increased by increasing the substrate concentration). (FIG. 5A) shows the simulation and (FIG. 5B) shows the experimental results obtained.

FIGS. 6A and 6B illustrates that optimizing the gain of this specific one-pot assay can be achieved by decreasing k₃ (i.e. increases the 36-fold difference between k₂ and k₃-see FIG. 9). Here, k₃ was decreased by increasing the concentration of the anchoring DNA on the surface of the electrode (hybridization rates were decreased when increasing charge repulsion). (FIG. 6A) shows the simulation and (FIG. 6B) shows the experimental results obtained).

FIGS. 7A to 7D provide an investigation of different complementary length of signaling DNA with aptamer on quinine (SEQ ID NO: 1, FIG. 7A) and thrombin (SEQ ID NO: 10, FIG. 7B) assays. Dose-response curves of the quinine (FIG. 7C) and thrombin (FIG. 7D) one-pot detection assays. Error bars show the standard deviation of three experiments.

FIGS. 8A to 8D demonstrate the multiplexing ability of this assay. (FIG. 8A) presents the above mentioned one-pot kinetic-based assay for quinine detection (signal-on assay). (FIG. 8B) presents an electrochemical steric-hindrance hybridization assay (eSHHA) for antibody detection (signal-off assay) (Mahshid et al., 2015). (FIGS. 8C, D) Two electrodes, each functionalized with a specific anchoring DNA are used to detect quinine (right-ended bars) and antibody (left-ended bars) simultaneously in whole blood. Error bars show the standard deviation of three experiments.

FIG. 9 illustrates the kinetic traces of the three different reactions. Association kinetic for the Quinine-Aptamer complex (25 s⁻¹). K₂: Association kinetic for the Aptamer-Signaling complex (0.022 s⁻¹). K₃: Association kinetic for the Signaling-Anchoring complex (0.00061 s⁻¹).

FIG. 10 illustrates the hybridization kinetics between the signaling DNA-16 (100 nM) and the surface-attached anchoring DNA with different complementary length (12, 14, and 16). Signaling DNA-16: MB-5′-ATT TTC CTT GTC TCC C-3′ (SEQ ID NO: 4); Anchoring DNA-16d: 5′-GGG AGA CAA GGA AAA T-3′-SH (SEQ ID NO: 8); Anchoring DNA-14d: 5′-G AGA CAA GGA AAA T-3′-SH (SEQ ID NO: 21) and Anchoring DNA-12d: 5′-GA CAA GGA AAA T-3′-SH (SEQ ID NO: 22).

FIG. 11 illustrates the hybridization kinetics between a fluorophore/quencher labeled quinine aptamer and unlabeled signaling DNA of different complementary length (12, 14, and 16). FAM-quinine aptamer-BHQ: FAM-5′-GGG AGA CAA GGA AAA TCC TTC AAT GAA GTG GGT CGA CA-3′-BHQ (SEQ ID NO: 1); Signaling DNA-12u: 5′-G AAA TCC TTG TCT CCC-3′ (SEQ ID NO: 2); Signaling DNA-14u: 5′-G ATT TCC TTG TCT CCC-3′ (SEQ ID NO: 3) and Signaling DNA-16u: 5′-A TTT TCC TTG TCT CCC-3′ (SEQ ID NO: 4).

FIG. 12 illustrates that the addition of quinine to the electrochemical one-pot reaction did not modify the electrochemical current in absence of quinine-binding aptamer.

FIGS. 13A to 13C illustrate the (FIG. 13A) binding curve of quinine and aptamer (Kd₁=386 nM); (FIG. 13B) binding curve of aptamer and signaling DNA (Kd₂=17 nM); (FIG. 13C) binding curve of signaling DNA and anchoring DNA (Kd₃=104 nM).

### DETAILED DESCRIPTION

In accordance with the present disclosure, there is provided a system as well as a method for detecting a target comprising three populations of distinct and unimolecular oligonucleotides: anchoring oligonucleotides, signaling oligonucleotides and targeting oligonucleotides. In the systems and methods of the present disclosure, anchoring oligonucleotides are associated with a substrate (also referred to herein as a ligand) and are designed to hybridize with signaling oligonucleotides which have not hybridized with targeting oligonucleotides. The role of the anchoring oligonucleotides is to locate, in the presence of the target, signaling oligonucleotides at the surface of the substrate. Signaling oligonucleotides include a reporter moiety which, when located at the vicinity of the surface of the substrate, triggers a detectable signal in combination with the substrate. The role of the signaling oligonucleotides is, when hybridized with the anchoring oligonucleotides, to allow the generation of a detectable signal thereby allowing the detection and, in some embodiments, the quantification, of the target in the sample being analyzed. The system of the present disclosure also includes targeting oligonucleotides which are designed to hybridize, in the absence of the target, to the signaling oligonucleotides so as to inhibit their association with the anchoring oligonucleotides (and ultimately prevent the generation of the detectable signal at the surface of the substrate). In the presence of the target, the targeting oligonucleotides are designed to bind specifically to the target, thus precluding their association to the signaling oligonucleotides allowing them to hybridize with the anchoring oligonucleotides and locate at the surface of the substrate. The role of the targeting oligonucleotides is thus to inhibit the association between the signaling and anchoring oligonucleotides in the absence of the target and to allow the association between the signaling and anchoring oligonucleotides in the presence of the target.

The systems and methods of the present disclosure use kinetically programmed oligonucleotides in order to perform a kinetically controlled physico-chemical reaction. As used in the present disclosure, the expression “kinetically controlled” refers to a physico-chemical reaction which is driven to first reach a kinetically accessible state and not an equilibrium. By the same token, the expression “kinetically programmed” refers to the properties of the different components of the systems or the steps of the methods which achieve kinetic control. In the present disclosure, the physico-chemical reaction is controlled by three distinct association constants: a first association constant (k₁) between the targeting oligonucleotide and the target, a second association constant (k₂) between the targeting oligonucleotide and the signaling oligonucleotide and a third association constant (k₃) between the signaling oligonucleotide and an anchoring nucleotide. The components of the system are kinetically programmed and the steps of the method are conducted so as to achieve a kinetically controlled physico-chemical reaction in which k₁>k₂>k₃ In an embodiment, the system is configured and the method is performed such that k₁ is at least 10 times and in some additional embodiments at least 100 times or 1000 times higher than k₂. Alternatively or in combination, the system is configured and the method is performed such that k₂ is at least 10 times and in some additional embodiments at least 100 times or 1000 times higher than k₃.

In certain embodiments, the detection system and method described herein are capable of specifically identifying millimolar, micromolar, nanomolar or picomolar concentrations of targets in a sample.

In certain embodiments, the detection system and method described herein are capable of detecting and optionally quantifying the target in the sample in less than an hour, for example in less than 55, 50, 45, 40, 35, 30, 25, 20, 15, 10, 9, 8, 7, 6, 5, 4, 3, 2 or 1 minutes or less.

The systems and methods described herein are particularly advantageous as they are designed to provide a detectable signal in the presence of the target and no detectable signal above a background noise in the absence of the target (“signal on” design). In some embodiments of the present disclosure, the systems and methods provides a similar signal gain for each targets to be detected irrespective of the type of target(s) being detected. In additional embodiments of the present disclosure, the systems and methods can be designed to accommodate the detection of target in a complex mixture (such as whole blood) and/or a plurality of targets in a mixture (e.g., multiplex detection). In further embodiments, the systems and methods described herein can be designed so as to allow the detection of the target (or the combination of targets) in a single reaction vessel (e.g., “one pot” reaction).

In addition, the systems and methods described herein are particularly advantageous over those described in WO2015/149184 as they are designed to provide a detectable signal in the presence of the target and no detectable signal above a background noise in the absence of the target (“signal on” design). In addition, in some embodiments, the signaling oligonucleotide of the present disclosure can be shorter and/or easier/cheaper to manufacture as those described in WO2015/149184 as it does not require (e.g., it lacks) a moiety for binding a macromolecular entity. In addition, the systems and methods described herein do not rely on the presence of steric hindrance at the surface of the substrate to determine the presence and optionally quantify the target in the sample.

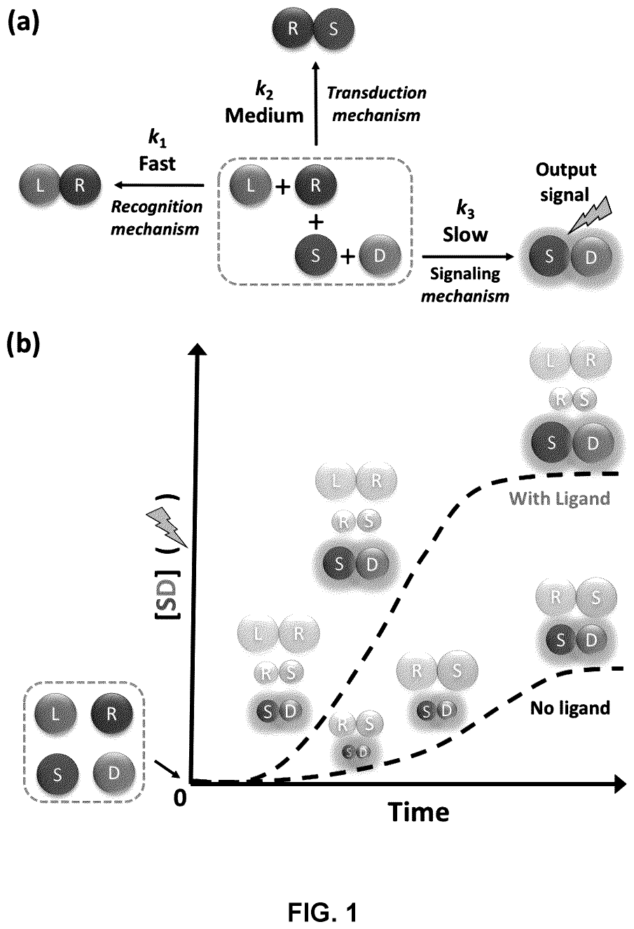

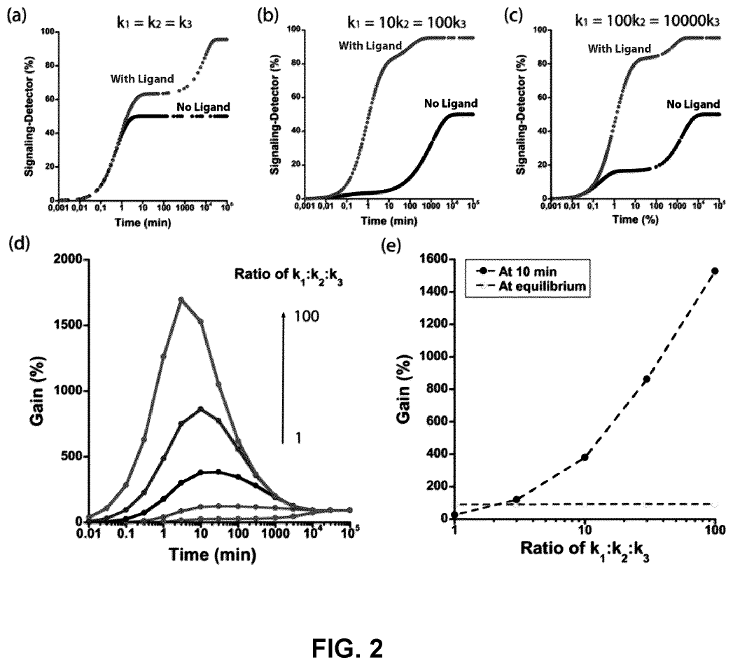

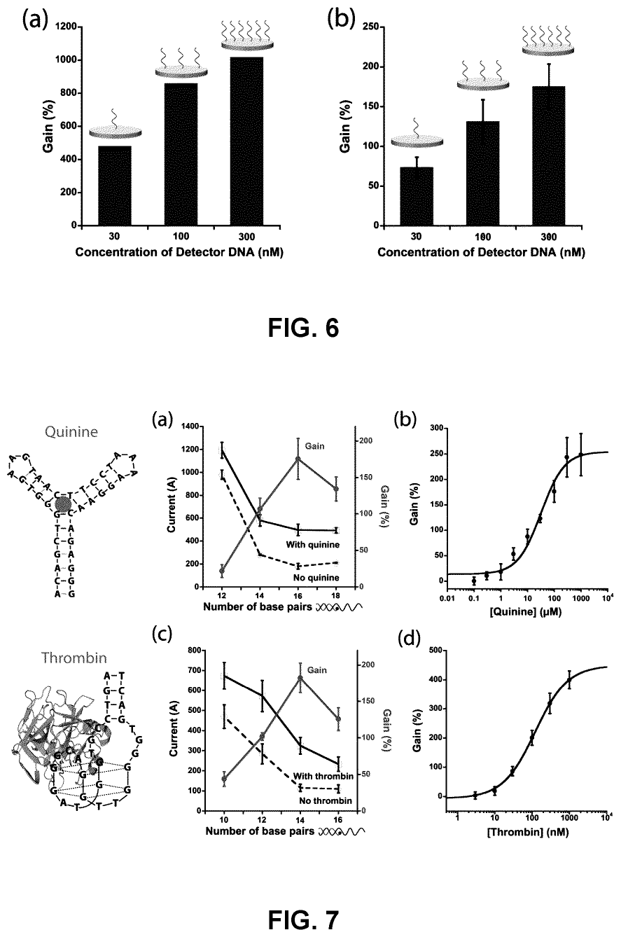

In an embodiment of the present disclosure, a universal kinetically-controlled “one-pot” three-reaction procedure is proposed in which the concentration of a specific target molecule, T, controls the yield of a convenient, signaling reaction (FIG. 1). In this strategy, the recognition mechanism, reaction 1, is achieved via a receptor molecule, R (referred herein as the targeting oligonucleotide). The presence of a specific ligand is then transduced via a transduction mechanism (reaction 2): upon binding to its ligand, the receptor is no longer able to bind to the signaling molecule (referred herein as the signaling oligonucleotide). If free, the signaling molecule can then interact with the anchoring molecule (referred herein as the anchoring oligonucleotide) to generate a signal output proportional to the ligand concentration. This strategy possesses two main advantages. First, the design principle is universal for all ligands for which we possess simple receptors such as DNA or RNA aptamer or somamers and it requires little (if no) optimization: the signaling mechanisms can be similar even for aptamer or ligand with very different structures (see for example FIG. 7). Second, this sensor can be kinetically programmed to generate high gains rapidly, well before reaching equilibrium (see FIG. 2).

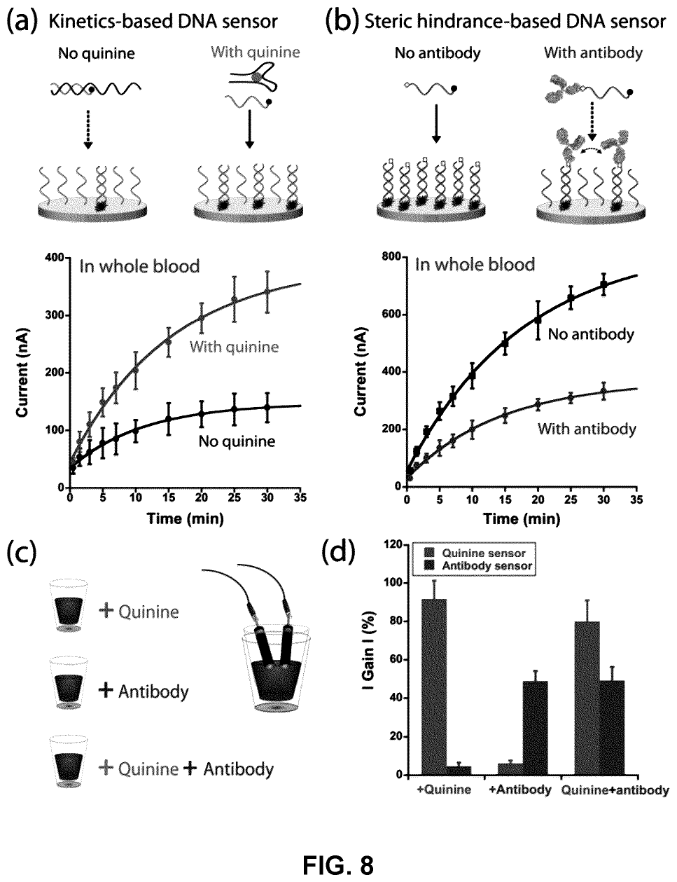

The molecular sensing mechanism system can be selected to specifically recognize a wide range of molecules and can be readily programmed to create efficient one-pot reactions. For example, specific DNA sequences can be selected to only react with their intended target (no side product) in a good yield. Another advantage of DNA is that multiple DNA reactions can proceed simultaneously in a unique complex sample (e.g., whole blood) without side reactions thus enabling multiplexing detection (see FIG. 8). Third, an often overlooked aspect of “one pot” reaction is that the kinetic of sequential reactions must be appropriately tuned so that each reaction takes place in time at the right moment. To this end, DNA hybridization can be tuned both thermodynamically and kinetically. Finally, DNA receptors can be re-engineer to signal the presence of an analyte molecule through various simple, universal allosteric DNA-based mechanism.

### Kinetically Programed Systems

As indicated above, the systems of the present disclosure include a plurality (e.g., a population) of anchoring oligonucleotides. In the detection system described herein, the anchoring oligonucleotide is capable of localizing the signaling oligonucleotide near the surface of the substrate so as to allow the formation of a detectable signal when the target is present. This is achieved by designing the anchoring oligonucleotide in such a way that it is capable of hybridizing (via Watson-Crick pairing) with the signaling oligonucleotide in the presence of the target in the system. Still in the detection system described herein, the anchoring oligonucleotide is not free to diffuse in solution, it associated (and, in an embodiment, covalently associated) to the surface of the substrate.

The anchoring oligonucleotide can also be referred to as a detector oligonucleotide or a capturing oligonucleotide. In the context of the present disclosure, the anchoring oligonucleotide is an oligonucleotide, preferably a unimolecular single-stranded and linear oligonucleotide, which is associated at one of its end (referred to the “first” end) to the surface of the substrate. Even though the anchoring oligonucleotide is provided as a unimolecular oligonucleotide, it is capable of hybridizing to the signaling oligonucleotide. It is contemplated that the anchoring oligonucleotide be associated to the surface of the substrate either via a terminal nucleic acid base (e.g., its 5′ or 3′ nucleic acid terminus) or via an internal nucleic acid base, preferably located within the five nucleic acid bases adjacent to the 5′ or 3′ nucleic acid terminus of the anchoring nucleotide. In an embodiment, the anchoring oligonucleotide is attached via its first end in a covalent manner to the surface of the substrate. The anchoring oligonucleotide can be associated directly to surface of the substrate or, alternatively, can be associated to the surface of the substrate through the use of a linker. The other end of the anchoring oligonucleotide (referred to as the “second” end) is considered “free” because it is not attached directly to the surface of the substrate. The anchoring oligonucleotide is configured such that a region (referred to as a “first region”) is exposed and is being relatively free to hybridize with the signaling oligonucleotide.

In embodiments of the system in which the anchoring oligonucleotide is associated to the surface of the substrate indirectly via a linker, it is contemplated that the linker can be any linker which will allow the positioning of the reporter moiety of the signaling oligonucleotide close to the surface of the substrate upon the hybridizing of the anchoring oligonucleotide with the signaling oligonucleotide. In an embodiment, the linker moiety may include 1 to 25 carbon atoms, such as 2 to 20 carbon atoms, including 5 to 15 carbon atoms. Exemplary embodiments of the linker include, but are not limited to, alkyl, preferably a lower straight-chain alkyl (e.g., C₁ to C₁₀) and even more preferably a C₁-C₆ alkyl. In some embodiments, the linker is a C₆ straight-chain alkyl.

The anchoring oligonucleotide is composed of any combination of known natural or synthetic nucleic acid bases and its backbone can be modified from naturally-occurring backbones. The anchoring oligonucleotide can be exclusively made from DNA or from RNA or can include both DNA and RNA. In an embodiment, the anchoring oligonucleotide is composed exclusively of DNA. Naturally-occurring oligonucleotides contain phosphodiester bonds and synthetic oligonucleotides comprising nucleic acid analogs may have alternate backbones, comprising, for example, phosphoramide, phosphorothioate, phosphorodithioate, O-methylphosphoroamidite linkages and peptide nucleic acid backbones and linkages. Other analog nucleic acids analogs include those with positive backbones, non-ionic backbones, and non-ribose backbones. Nucleic acids bases containing one or more carbocyclic sugars are also included within the definition of contemplated nucleic acid bases.

The anchoring oligonucleotide generally has a total length between 10 and 30 nucleic acid bases. The length and composition (GC and AT content) of the anchoring oligonucleotide is designed in order to achieve a sufficiently good affinity between the anchoring and signaling oligonucleotides (for example, a K_{D} of at least 10 nM). The anchoring oligonucleotide can have a total length of at least 10, 11, 12, 13, 14, 15, 16, 17, 18, 19, 20, 21, 22, 23, 24, 25, 26, 27, 28, 29 or 30 nucleic acid bases and/or a total length of no more than 30, 29, 28, 27, 26, 25, 24, 23, 22, 21, 20, 19, 18, 17, 16, 15, 14, 13, 12, 11 or 10 nucleic acid bases. In an embodiment, the anchoring oligonucleotide has a total length between 12 and 18 nucleic acid bases.

The anchoring oligonucleotide also comprises a region (referred to a first region) designed for hybridizing to a first nucleic acid sequence of the signaling oligonucleotide. In an embodiment, the first region can encompass the entire length of the anchoring oligonucleotide. In another embodiment, the anchoring oligonucleotide can include additional nucleic acid bases (located 3′ and/or 5′ to the first region) which do not hybridize to the first nucleic acid sequence of the signaling oligonucleotide. For example, the first region of the anchoring oligonucleotide can be at least 8, 9, 10, 11, 12, 13, 14, 15, 16, 17, 18, 19, 20, 21, 22, 23, 24, 25, 26, 27, 28, 29 or 30 and/or no more than 30, 29, 28, 27, 26, 25, 24, 23, 22, 21, 20, 19, 18, 17, 16, 15, 14, 13, 12, 11, 10, 9 or 8 contiguous nucleic acid bases long. The level of complementarity between the first region of the anchoring oligonucleotide and the first nucleic acid sequence of the signaling oligonucleotide is substantially identical and can be at least 80%, 85%, 90%, 91%, 92%, 93%, 94%, 95%, 96%, 97%, 98%, 99% or 100%. In one embodiment, the entire length of the anchoring oligonucleotide is complementary to the first nucleic acid sequence of the signaling oligonucleotide. In another embodiment, the anchoring oligonucleotide or the first region of the anchoring oligonucleotide is complementary over the entire length of the first nucleic acid sequence of the signaling oligonucleotide.

In the system and methods of the present disclosure, the anchoring oligonucleotide is designed in such as way so as to compete with the targeting oligonucleotide for hybridizing with the signaling oligonucleotide. This can be achieved by including in the anchoring oligonucleotide a first region (as described above) which is substantially identical to a second region present in the signaling oligonucleotide. For example, the first region of the anchoring oligonucleotide which is substantially identical to the second region of the targeting oligonucleotide can be at least 8, 9, 10, 11, 12, 13, 14, 15, 16, 17, 18, 19 or 20 and/or no more than 20, 19, 18, 17, 16, 15, 14, 13, 12, 11, 10, 9 or 8 contiguous nucleic acid bases long. In another example, the second region of the targeting oligonucleotide which is substantially identical to the second region of the targeting oligonucleotide can be at least 8, 9, 10, 11, 12, 13, 14, 15, 16, 17, 18, 19, or 20 and/or no more than 20, 19, 18, 17, 16, 15, 14, 13, 12, 11, 10, 9 or 8 contiguous nucleic acid bases long. The level of identity between the first region of the anchoring oligonucleotide and the second region of the targeting oligonucleotide can be, for example, at least 80%, 85%, 90%, 91%, 92%, 93%, 94%, 95%, 96%, 97%, 98%, 99% or 100%. This level of identity can be achieved over the entire length of the first region and/or over the entire length of the second region. The anchoring oligonucleotide can include one or more additional nucleic acid bases (located 3′ and/or 5′ to the first region) which are not identical to the second region of the targeting oligonucleotide.

In systems using a substrate (such as a gold electrode) having a surface area of 0.0314 cm², the anchoring oligonucleotide can be attached on the surface by employing a concentration of at least 10, 20, 30, 40, 50, 60, 70, 80, 90, 100, 125, 150, 175, 200, 225, 250, 275, 300, 325, 350, 365, 400, 425, 450, 475, 500 nM or more. Alternatively or in combination, still in systems using a substrate having a surface area of 0.0314 cm², the anchoring oligonucleotide can be provided at a concentration of no more than 500, 475, 450, 425, 400, 375, 350, 325, 300, 275, 250, 225, 200, 175, 150, 125, 100, 90, 80, 70, 60, 50, 40, 30, 20, 10 nM or less. In still additional embodiments in systems using a substrate having a surface area of 0.0314 cm², the anchoring oligonucleotide can be provided at a concentration between about 10, 20, 30, 40, 50, 60, 70, 80, 90, 100, 125, 150, 175, 200, 225, 250, 275, 300, 325, 350, 365, 400, 425, 450 or 475 nM and about 500, 475, 450, 425, 400, 375, 350, 325, 300, 275, 250, 225, 200, 175, 150, 125, 100, 90, 80, 70, 60, 50, 40, 30 or 20 nM. In still another embodiment, the anchoring oligonucleotide can be provided at a concentration between about 30 and 300 nM (in systems using a substrate having a surface area of 0.0314 cm²).

In order to provide more kinetic control over the physico-chemical reaction, the system can be designed in order to reduce the speed at which the signaling oligonucleotide hybridizes to the anchoring oligonucleotide, to ultimately reduce k₃. For example, the anchoring oligonucleotide can be attached at higher density on the surface (using more concentrated anchoring oligonucleotide solution or using electrodeposition methods), which reduces the hybridization rate of the signaling oligonucleotide due to increase charge repulsion. The anchoring oligonucleotide can also be lengthened (e.g., equal to or higher than 12, 14, 16 or 18 nucleic acid bases) or can be adjusted to be longer than the length of the signaling oligonucleotide. In another example, the anchoring oligonucleotide can be provided in a conformation in which part of the anchoring oligonucleotide is double stranded, for example by allowing the formation of a hairpin structure close or in the first region of the anchoring oligonucleotide. Alternatively or in combination, the system can include an additional oligonucleotide capable of hybridizing in the vicinity or close to the first region of the anchoring oligonucleotide so as to reduce k₃.

As indicated above, the system of the present disclosure also comprises a signaling oligonucleotide. In some embodiments, the signaling oligonucleotide is a unimolecular single-stranded and linear oligonucleotide. Even though the signaling oligonucleotide is unimolecular, it can bind to the anchoring oligonucleotide as well as to the targeting oligonucleotide in a competitive manner. The signaling oligonucleotide is associated to, at one end, a reporter moiety (that can be covalently attached to one signaling oligonucleotide). In the present disclosure, the signaling oligonucleotide comprises a first nucleic acid sequence (for hybridizing to the first region of the anchoring oligonucleotide) and a second nucleic acid sequence (for hybridizing to a second region of a targeting oligonucleotide). The first and second nucleic acid sequences can refer to the same or a different nucleic acid sequences. Still in the context of the detection system described herein, the signaling oligonucleotide has the ability to diffuse in solution, it is not necessarily associated to the surface of the substrate but can nevertheless hybridizes with an anchoring oligonucleotide and localize at the surface of the substrate (especially in the presence of the target).

The signaling oligonucleotide generally has a total length between 10 and 30 nucleic acid bases. The length and composition (GC and AT content) of the signaling oligonucleotide is designed in order to achieve a sufficiently good affinity between the anchoring and signaling oligonucleotides (for example, a K_{D} of at least 10 nM). The signaling oligonucleotide can have a total length of at least 8, 9, 10, 11, 12, 13, 14, 15, 16, 17, 18, 19, 20, 21, 22, 23, 24, 25, 26, 27, 28, 29 or 30 nucleic acid bases and/or a total length of no more than 30, 29, 28, 27, 26, 25, 24, 23, 22, 21, 20, 19, 18, 17, 16, 15, 14, 13, 12, 11, 10, 9 or 8 nucleic acid bases. In an embodiment, the signaling oligonucleotide has a total length between 12 and 18 nucleic acid bases.

It is important that the signaling oligonucleotide is not capable of binding simultaneously to both the anchoring oligonucleotide and to the targeting oligonucleotide to allow the competition between the anchoring oligonucleotide and the targeting oligonucleotide for binding to the signaling oligonucleotide. This can be done by selecting for a first and a sequence nucleic acid sequences and including same in the signaling oligonucleotide for competitively binding to the anchoring and the targeting oligonucleotide.

The signaling oligonucleotide comprises a nucleic acid sequence (referred to as a first nucleic acid sequence) designed for hybridizing to a first region of the anchoring oligonucleotide. In an embodiment, the first nucleic acid sequence can encompass the entire length of the signaling oligonucleotide. In another embodiment, the signaling oligonucleotide can include additional nucleic acid bases (located 3′ and/or 5′ to the first region) which do not hybridize to the first region of the anchoring oligonucleotide (but which can, in some embodiments, hybridize to the second region of the targeting oligonucleotide). For example, the first nucleic acid sequence of the signaling oligonucleotide can be at least 8, 9, 10, 11, 12, 13, 14, 15, 16, 17, 18, 19 or 20 and/or no more than 20, 19, 18, 17, 16, 15, 14, 13, 12, 11, 10, 9 or 8 contiguous nucleic acid bases long. The level of complementarity between the first nucleic acid sequence of the signaling oligonucleotide and the corresponding first region of the anchoring oligonucleotide is substantially identical and can be at least 80%, 85%, 90%, 91%, 92%, 93%, 94%, 95%, 96%, 97%, 98%, 99% or 100%. In one embodiment, the entire length of the signaling oligonucleotide is complementary to the corresponding second region of the anchoring oligonucleotide. In another embodiment, the signaling oligonucleotide or the first nucleic acid sequence of the signaling oligonucleotide is complementary over the entire length of the first region of the anchoring oligonucleotide.

The signaling oligonucleotide also comprises a nucleic acid sequence (referred to as a second nucleic acid sequence) designed for hybridizing to a second region of the targeting oligonucleotide. In an embodiment, the second nucleic acid sequence can encompass the entire length of the signaling oligonucleotide. In another embodiment, the signaling oligonucleotide can include additional nucleic acid bases (located 3′ and/or 5′ to the first region) which do not hybridize to the second region of the targeting oligonucleotide (but which can, in some embodiments, hybridize to the first region of the anchoring oligonucleotide). For example, the second nucleic acid sequence of the signaling oligonucleotide can be at least 8, 9, 10, 11, 12, 13, 14, 15, 16, 17, 18, 19 or 20 and/or no more than 20, 19, 18, 17, 16, 15, 14, 13, 12, 11, 10, 9 or 8 contiguous nucleic acid bases long. The level of complementarity between the second nucleic acid sequence of the signaling oligonucleotide and the corresponding second region of the targeting oligonucleotide is substantially identical and can be at least 80%, 85%, 90%, 91%, 92%, 93%, 94%, 95%, 96%, 97%, 98%, 99% or 100%. In one embodiment, the entire length of the signaling oligonucleotide is complementary to the corresponding second region of the anchoring oligonucleotide. In another embodiment, the signaling oligonucleotide or the second nucleic acid sequence of the signaling oligonucleotide is complementary over the entire length of the second region of the targeting oligonucleotide.

The signaling oligonucleotides described herein are composed of any combinations of known natural or synthetic nucleic acid bases and its backbone can be modified from naturally-occurring backbones. The signaling oligonucleotide can be exclusively made from DNA or from RNA or can include both DNA and RNA. In an embodiment, the signaling oligonucleotide can be exclusively made from DNA. Naturally-occurring oligonucleotides contain phosphodiester bonds and synthetic oligonucleotides comprising nucleic acid analogs may have alternate backbones, comprising, for example, phosphoramide, phosphorothioate, phosphorodithioate, O-methylphosphoroamidite linkages and peptide nucleic acid backbones and linkages. Other analog nucleic acids analogs include those with positive backbones, non-ionic backbones, and non-ribose backbones. Nucleic acids bases containing one or more carbocyclic sugars are also included within the definition of contemplated nucleic acid bases.

It is contemplated that the signaling oligonucleotide be associated with the reporter moiety either via a terminal nucleic acid base (e.g., its 5′ or 3′ nucleic acid terminus) or via an internal nucleic acid base, preferably located within the five nucleic acid bases adjacent to the 5′ or 3′ nucleic acid terminus of the signaling oligonucleotide. In an embodiment, the signaling oligonucleotide is attached via its first end in a covalent manner to the reporter moiety. The signaling oligonucleotide can be associated directly to reporter moiety or, alternatively, can be associated to the reporter moiety through the use of a linker. In embodiments of the system in which the signaling oligonucleotide is associated to reporter moiety indirectly via a linker, it is contemplated that the linker can be any linker which will allow the location of the reporter moiety in the vicinity of the surface of the substrate when the anchoring and the signaling oligonucleotides are hybridized. In an embodiment, the linker moiety may include 1 to 25 carbon atoms, such as 2 to 20 carbon atoms, including 5 to 15 carbon atoms. In an embodiment, the linker is a alkyl, such as a straight chain lower alkyl (e.g., C₁ to C₁₀ lower alkyl), and preferably a C₃ to C₆ alkyl. In some embodiments, the linker is a C₆ straight-chain alkyl.

The signaling oligonucleotide can be configured so as to avoid or limit hybridization with the targeting oligonucleotide in the presence of the target (e.g., to decrease k₂ drastically when the target is bound to the aptamer). This can be done, for example, by designing the signaling oligonucleotide in such a way that it binds to the second region of the targeting oligonucleotide which is base-paired or non-accessible in the presence of the target and/or directly involved in contacting the target. The signaling oligonucleotide can be also configured so as to increase its hybridization rate with the targeting oligonucleotide in absence of the target (e.g., to increase k₂ in absence of target). This can be done, for example, by designing the signaling oligonucleotide in such a way that it binds to the second region of the targeting oligonucleotide that is accessible to the solvent (or accessible to binding) when the aptamer is folded in absence of target.

The system described herein also include a plurality of targeting oligonucleotides. The targeting oligonucleotide is an oligonucleotide, preferably a unimolecular and single-stranded oligonucleotide, which is capable of specifically binding to the target as well as to the signaling oligonucleotide. The targeting oligonucleotides is configured such as to preferably bind to the target instead of the signaling oligonucleotide. The targeting oligonucleotide can diffuse in the system and is not associated with the surface of the substrate (e.g., it has two “free” ends). In some embodiments, the targeting oligonucleotide can include one or more hairpin structures involved in binding the target. However, in such embodiments, the targeting oligonucleotide also includes a second region (usually single-stranded) which is accessible for binding to the second sequence of the signaling oligonucleotide.

The targeting oligonucleotide is composed of any combination of known natural or synthetic nucleic acid bases and its backbone can be modified from naturally-occurring backbones. The targeting oligonucleotide can be an aptamer (e.g., exclusively composed of naturally-occurring nucleic acid base or containing some or only non-naturally-occurring nucleic acid), a SOMAamer (e.g., having a very low dissociation rate and comprising naturally occurring as well as non-naturally-occurring nucleic acid bases) or a combination thereof. The targeting oligonucleotide can be exclusively made from DNA or from RNA or can include both DNA and RNA. In an embodiment, the targeting oligonucleotide is exclusively made from DNA. Naturally-occurring oligonucleotides contain phosphodiester bonds and synthetic oligonucleotides comprising nucleic acid analogs may have alternate backbones, comprising, for example, phosphoramide, phosphorothioate, phosphorodithioate, O-methylphosphoroamidite linkages and peptide nucleic acid backbones and linkages. Other analog nucleic acids analogs include those with positive backbones, non-ionic backbones, and non-ribose backbones. Nucleic acids bases containing one or more carbocyclic sugars are also included within the definition of contemplated nucleic acid bases.

The targeting oligonucleotide generally has a total length between 8 and 200 nucleic acid bases. The length and composition (GC and AT content) of the targeting oligonucleotide can be selected or is designed in order to achieve a sufficiently good affinity between the targeting and signaling oligonucleotides (for example, a K_{D} of 10 nM). The targeting oligonucleotide can have a total length of at least 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200 or more nucleic acid bases and/or a total length of no more than 200, 190, 180, 170, 160, 150, 140, 130, 120, 110, 100, 90, 80, 70, 60, 50, 40, 30, 29, 28, 27, 26, 25, 24, 23, 22, 21, 20, 19, 18, 17, 16, 15, 14, 13, 12, 11, 10, 9, 8 or less nucleic acid bases. In an embodiment, the targeting oligonucleotide has a total length between 8 and 200 nucleic acid bases.

The targeting oligonucleotide also comprises a region (referred to a second region) designed for hybridizing to a second nucleic acid sequence of the signaling oligonucleotide. In an embodiment, the second region can encompass the entire length of the targeting oligonucleotide. In another embodiment, the targeting oligonucleotide can include additional nucleic acid bases (located 3′ and/or 5′ to the second region) which do not hybridize to the second nucleic acid sequence of the signaling oligonucleotide. For example, the second region of the targeting oligonucleotide can be at least 8, 9, 10, 11, 12, 13, 14, 15, 16, 17, 18, 19, 20, 21, 22, 23, 24, or 25 and/or no more than 25, 24, 23, 22, 21, 20, 19, 18, 17, 16, 15, 14, 13, 12, 11, 10, 9 or 8 contiguous nucleic acid bases long. The level of complementarity between the second region of the targeting oligonucleotide and the second nucleic acid sequence of the signaling oligonucleotide is substantially identical and can be at least 80%, 85%, 90%, 91%, 92%, 93%, 94%, 95%, 96%, 97%, 98%, 99% or 100%. In one embodiment, the entire length of the targeting oligonucleotide is complementary to the second nucleic acid sequence of the signaling oligonucleotide. In another embodiment, the targeting oligonucleotide or the second region of the targeting oligonucleotide is complementary over the entire length of the second nucleic acid sequence of the signaling oligonucleotide.

In the system and methods of the present disclosure, the targeting oligonucleotide can be selected or designed in such as way so as to compete with the anchoring oligonucleotide for hybridizing with the signaling oligonucleotide. This can be achieved by including in the targeting oligonucleotide a second region (as described above) which is substantially identical to a first region present in the anchoring oligonucleotide. For example, the second region of the targeting oligonucleotide which is substantially identical to the first region of the anchoring oligonucleotide can be at least 8, 9, 10, 11, 12, 13, 14, 15, 16, 17, 18, 19, 20, 21, 22, 23, 24 or 25 and/or no more than 25, 24, 23, 22, 21, 20, 19, 18, 17, 16, 15, 14, 13, 12, 11, 10, 9 or 8 contiguous nucleic acid bases long. In another example, the second region of the targeting oligonucleotide which substantially identical to the first region of the anchoring oligonucleotide can be at least 8, 9, 10, 11, 12, 13, 14, 15, 16, 17, 18, 19, 21, 22, 23, 24 or 25 and/or no more than 25, 24, 23, 22, 21, 20, 19, 18, 17, 16, 15, 14, 13, 12, 11, 10, 9 or 8 contiguous nucleic acid bases long. The level of identity between the second region of the targeting oligonucleotide and the first region of the anchoring oligonucleotide can be, for example, at least 80%, 85%, 90%, 91%, 92%, 93%, 94%, 95%, 96%, 97%, 98%, 99% or 100%. This level of identity can be achieve over the entire length of the second region and/or over the entire length of the first region. The targeting oligonucleotide can include one or more additional nucleic acid bases (located 3′ and/or 5′ to the second region) which are not identical to the first region of the anchoring oligonucleotide.

In order to allow the kinetic control of the detection reaction to take place, the targeting oligonucleotide has a dissociation constant (K_{D}) with the target between 5 and 1000 times lower than the suspected concentration of the target in the sample being tested. In some embodiments, K_{D} is at least 5, 10, 20, 50, 100, 200, 300, 400, 500, 600, 700, 800, 900 or 1000 times lower than the suspected concentration of the target in the sample being tested. This feature favors the binding of the target to the targeting oligonucleotide (fast k₁), thus preventing the targeting oligonucleotide from hybridizing with the signaling oligonucleotide (slower k₂).

In order to avoid or limit the background noise associated with the system, the ratio between the molar concentration of the targeting oligonucleotide and the molar concentration the signaling oligonucleotide in the system can be adjusted to be equal to or more than 1.0.

Alternatively or in combination, the targeting oligonucleotide can be selected or configured so as to avoid or limit hybridization with the signaling oligonucleotide in the presence of the target (e.g., to favor k₁ instead of k₂). This can be done, for example, by selecting or designing the targeting oligonucleotide in such a way that the signaling oligonucleotide is intended to bind to region which is base-paired in the presence of the target or directly involved in contacting the target.

In embodiments in which it is warranted to increase the hybridization of the signaling oligonucleotide to the targeting oligonucleotide (such as, for example, to favor k₂ instead of k₃), it is possible to configure the targeting oligonucleotide to include additional nucleic acid bases which are not involved in the binding of the target but are complementary (at least in part) to the signaling oligonucleotide. For example, adding modified nucleotides with positive charge(s) to the end of the targeting oligonucleotide could increase k₂.

In some embodiments, the targeting oligonucleotide can be provided at a molar concentration which is higher than the molar concentration of the anchoring oligonucleotides so as to favor k₂ instead of k₃.

The system described herein include and the method described herein include a substrate. In the context of the present disclosure, the substrate provides a surface for associating with at least one anchoring oligonucleotide to prevent it from freely diffusing in solution/suspension. In close proximity to the reporter moiety of signaling oligonucleotide, the surface of the substrate is also capable of creating, modulating or conducting a signal upon the hybridizing of the signaling oligonucleotide with the anchoring oligonucleotide. For example, the surface of the substrate can be a fluorescent one and the reporter moiety on the signaling oligonucleotide can be a quencher moiety capable of limiting the fluorescence associated with the surface of the substrate. In such embodiment, upon the hybridizing of the anchoring oligonucleotide with the signaling oligonucleotide, the quencher moiety comes into close proximity with the fluorescent surface of the substrate and create a modulation (e.g., reduction) in fluorescence, which can be detected and quantified. In another example, the substrate is a metallic electrode (such as a gold electrode) and the reporter moiety is a redox-reporter (methylene blue for example). In such example, upon the hybridizing of the anchoring oligonucleotide with the signaling oligonucleotide, the redox-moiety will come into close contact with the gold electrode and create a modulation in the current which be detected and optionally quantified.

The substrate may be a fluorescent substrate (e.g., comprising a fluorophore or a plurality of fluorophores) and the reporter moiety of the signaling oligonucleotide can be a corresponding quencher moiety (or a combination of quencher moieties). Alternatively, the reporter moiety can be a fluorophore (or a combination of fluorophores) and the substrate may be a corresponding quencher (e.g., comprising a quencher or a plurality of quenchers). In these instances, in the presence of target a majority of the signaling oligonucleotides will hybridize with the anchoring oligonucleotide, the distance the fluorophore is held from the quencher is sufficient to minimize, suppress, or prevent the fluorophore from emitting a detectable signal. Alternatively, in the absence of the target or the macromolecular entity, less signaling oligonucleotides are able to localize at the surface of the substrate and the fluorophore can emit a detectable signal. The term “fluorophore” refers to any molecular entity that is capable of absorbing energy of a first wavelength and re-emit energy at a different second wavelength. The fluorophore may be synthetic or biological in nature, as known to those of skill in the art. More generally, any fluorophore can be used that is stable under assay conditions and that can be sufficiently suppressed when in close proximity to the quencher such that a significant change in the intensity of fluorescence of the fluorophore is detectable in response to target specifically binding the probe. Examples of suitable fluorophores include, but are not limited to CAL Fluor Red 610 (FR610; Biosearch Technologies, Novato, Calif.), fluorescein isothiocyanate, fluorescein, 6-carboxyfluorescein (6-FAM), rhodamine and rhodamine derivatives, coumarin and coumarin derivatives, cyanine and cyanine derivatives, Alexa Fluors™ (Molecular Probes, Eugene, Oreg.), DyLight Fluors (Thermo Fisher Scientific, Waltham, Mass.), and the like.

The term “quencher” may refer to a substance that absorbs excitation energy from a fluorophore and dissipates that energy as heat. The quencher may also absorb excitation energy from a fluorophore and dissipate that energy as re-emitted light at a different wavelength. Quenchers are used in conjunction with fluorophores, such that when the quencher is positioned adjacent the fluorophore or at a distance sufficiently close to the fluorophore, the emission of the fluorophore is suppressed. However, when the quencher is positioned away from the fluorophore or at a distance sufficiently far from the fluorophore, the emission of the fluorophore is not suppressed, such that a signal of the fluorophore is detectable. Alternatively, the quencher may include moieties that reduce the emission of the fluorophore via photoelectron transfer, resonance energy transfer or other quenching mechanisms. The quencher may also be replaced by a second fluorophore capable of resonance energy transfer, by a second fluorophore capable of forming an excimer or exciplex or, in general, by any other group that modulates the fluorescence of the first fluorophore. The quencher may be synthetic or biological in nature, as known to those of skill in the art. More generally, any quencher can be used that is stable under assay conditions and that can sufficiently suppress the fluorescence of the fluorophore when in close proximity to the fluorophore such that a significant change in the intensity of fluorescence of the fluorophore is detectable in response to target/macromolecular entity specifically binding the signaling oligonucleotide. Examples of quenchers include, but are not limited to, Black Hole Quencher (BHQ; Biosearch Technologies, Novato, Calif.), Dabsyl (dimethylaminoazosulphonic acid), Qxl quenchers (AnaSpec Inc., San Jose, Calif.), Iowa black FQ, Iowa black RQ, and the like. In another embodiment the quencher may also be fluorescent, leading to emission at a second wavelength when the quencher is in proximity to the first fluorophore. Examples of such fluorophore/quencher pairs include Alexa488™-Alexa555™, Alexa488™-Cy3™, Cy3™-Cy5™. In other embodiments, the quencher is a second fluorophore that forms an excimer or an exciplex with the first fluorophore, leading to a change in fluorescence upon their segregation. An example would include an embodiment in which both the fluorophore and the quencher are pyrene.

In certain embodiments, the substrate and the reporter moiety of the present disclosure include a first signaling moiety that includes a macromolecule having a catalytic activity and a second signaling moiety that includes an inhibitor or an activator of the catalytic activity. In certain embodiments, the catalytic macromolecule is held at distance in close proximity to the inhibitor, such as adjacent the inhibitor, by complementary hybridizing of the anchoring and signaling oligonucleotides.

In still another embodiment, the substrate is a metallic electrode (such as a gold, silver, platinum) or a non-metallic electrode (e.g., carbon or silicon for example). The conductive and semiconductive materials can be metallic or non-metallic.) and the reporter moiety is a redox reporter (e.g., organic redox moieties, such as viologen, anthraquinone, ethidium bromide, daunomycin, methylene blue, and their derivatives, organo-metallic redox moieties, such as ferrocene, ruthenium, bis-pyridine, tris-pyridine, bis-imidazole, and their derivatives,

The anchoring oligonucleotides are associated on the surface of the substrate at various discrete positions. The anchoring oligonucleotides are configured on the surface of the substrate at a density which would allow a kinetic control of the detection of the target. The substrate described herein can be an “array”, a term which include any one-dimensional, two-dimensional or substantially two-dimensional (as well as a three-dimensional) arrangement of addressable regions bearing an anchoring oligonucleotide associated with that region. In some embodiments, the array can be tri-dimensional, such as for example a nanoparticle, such as a gold nanoparticle or a fluorescent nanoparticle. The substrate can be substantially planar or can take aspheric form. An “addressable array” includes any one or two dimensional arrangement of discrete regions bearing particular anchoring oligonucleotides associated with that region and positioned at particular predetermined locations on the substrate (each such location being at a known “address”). These regions may or may not be separated by intervening spaces. Any given substrate may carry one, two, four or more arrays disposed on a front surface of the substrate. Depending upon the use, any or all of the arrays may be the same or different from one another and each may contain multiple spots or features. A typical array may contain more than ten, more than one hundred, more than one thousand, more than ten thousand features, or even more than one hundred thousand features, in an area of less than 20 cm², such as less than 10 cm². For example, features may have widths (that is, diameter, for a round spot) in the range from a 10 μm to 1.0 cm. In other embodiments each feature may have a width in the range of 1.0 μm to 1.0 mm, such as 5.0 μm to 500 μm, including 10 μm to 200 μm. Non-round features may have area ranges equivalent to that of circular features with the foregoing width (diameter) ranges.

The systems and methods described herein are for the detection, and optionally the quantification, of one or more targets in a sample. In the context of the present disclosure, a “sample” is mixture (either already in a liquid or capable of being provided as in a liquid form) suspected of containing the target or the combination of targets of interest. The sample can be a solution or a suspension. The sample can be processed into a solution or a suspension. The sample can be a biological sample. Exemplary biological samples include, but are not limited to, bodily fluids (e.g., blood, blood components (such as plasma), urine, gastro-intestinal juice, interstitial fluid, lachrymal fluid, sweat, saliva, stools, sputum, pus, cerebrospinal fluid, semen, prostatic fluid, milk, nipple aspirate fluid, lachrymal fluid, perspiration), tissues (swabs (e.g., cheek swabs), tissue biopsy), fractionated bodily fluids (serum, plasma, etc.), cells, cell extracts (e.g., cytoplasmic membrane, mitochondrial extract, nuclear extracts, etc.), cell suspensions, secretions as well as cultures of such biological samples. The sample can be an environmental sample, such as, for example, a water, a gas sample or a soil sample. The sample can also be a food sample. In one embodiment, the sample can be contacted directly with the systems described herein or submitted directly to the methods described herein. In another embodiment, the sample can be treated (e.g., by adding or removing components to the sample) prior to contacting the system or being submitted to the methods described herein.

The system presented herewith is used for the detection of a target. As used in the context of the present disclosure, the “target” maybe any molecule of interest which is suspected to be present in a sample to be analysed and is capable of specifically binding to the targeting oligonucleotide. For example, the target can be an ions, cations, small molecule, a metabolite, a biologically-created entity such as a peptide, a polypeptide, a subcellular fraction, a cell, a cellular composition, a tissue, etc. In the context of the present disclosure, the expression “specific binding” or “specifically bind” refers to the interaction between two elements in a manner that is determinative of the presence of the elements in the presence or absence of a heterogeneous population of molecules that may include nucleic acids, proteins, and other biological molecules. For example, under designated conditions, a target binds to a particular targeting oligonucleotide and does not bind in a significant manner to other molecules in the sample or the system. The target preferably only binds to the targeting oligonucleotides and does not substantially bind to the signaling oligonucleotide, the anchoring oligonucleotide and/or to the substrate. In some embodiments, the substrate can be treated prior to contacting the sample suspected of containing the target so as to avoid or limit the binding of the target to the substrate.

The system described herein can be designed so as to detect, in a single reaction vessel, the presence or absence of the target. The system can be provided as a kit including at least one of a plurality of targeting oligonucleotides, a plurality of signaling oligonucleotides, at least one substrate associated with a plurality of anchoring oligonucleotides and instructions to perform the detection or the quantification of the target. The kit can also include a buffer, a source of cations, and control amounts for each of the targets, a positive control and/or a negative control. A positive control of the kits of the present disclosure can include, for example, a control signaling oligonucleotide capable of hybridizing to a control anchoring oligonucleotide and lacking a corresponding targeting oligonucleotide. The use of positive control provides a strong signal even in the absence of the target and thus allows determining if the generation of the signal is workable in the system. A negative control of the kits of the present disclosure can include, for example, a control signaling oligonucleotide capable of hybridizing to a control anchoring oligonucleotide and a (diffuse) control oligonucleotide complementary to the control signaling oligonucleotide. The use of the negative control provides a very low or no signal even in the presence of the target since the control signaling oligonucleotide will hybridize to the control oligonucleotide instead of the corresponding anchoring oligonucleotide and thus allows determining the background noise (if any) of the system.

The system described herein can be provided to detect a plurality of distinct targets (e.g., multiplexing). In such embodiment, the system provide, for each distinct target, distinct and corresponding targeting, signaling and anchoring oligonucleotides. The anchoring oligonucleotides can be provided on distinct and corresponding substrates or arrayed at discrete locations on one or more substrates.

### Kinetically Programmed Methods

The present disclosure also provides a method of detecting and, in some embodiments, quantifying, a target in a sample. The method relies on using the components of a kinetically programmed system to achieve the kinetically control detection of the target or the plurality of targets. The method first includes providing a sample suspected or known to have the target. The sample can be directly submitted to the detection method or can be treated (components can be added to the sample or can be removed from the sample) prior to conducting the detection method. The method also includes providing the system as described herein which can allow the detection of one or more distinct targets using kinetically programmed components.

The method can be designed to provide an output (e.g., a gain) comparing a signal (which can be an electrochemical signal as indicated herein) generated at the surface of the one or more substrates in the presence (test amount) and in the absence (control amount) of the target. As such, the method thus comprises either providing or determining the signal associated with the hybridization of the signaling oligonucleotides and the anchoring oligonucleotides in the absence of the one or more targets (control amount). Since the present method is designed as a “signal-on” determination, this control amount is supposed to be minimal. The method can include referring to previous determinations made in corresponding systems.

The method also comprises contacting the sample with the system to provide the signal generated at the surface of the one or more substrates in the sample and presumably in the presence of the substrate (test amount). As such, the method thus comprises determining the signal associated with the hybridization of the signaling oligonucleotides and the anchoring oligonucleotides in the sample. In order to generate the test amount, the sample is first contacted with the targeting oligonucleotides and the signaling oligonucleotides in the absence of the anchoring oligonucleotides (to provide a targeted mixture). Optionally, the sample can first be contacted with the targeting oligonucleotides in the absence of the signaling oligonucleotides and then be contacted with the signaling oligonucleotides (to provide the targeted mixture). Alternatively, the sample can first be contacted with the signaling oligonucleotides in the absence of the targeting oligonucleotides and then be contact with the targeting oligonucleotides (to provide the targeted mixture). Then the targeted mixture is contacted with the anchoring oligonucleotides (to provide a detectable mixture) so as to allow the signaling oligonucleotides that did not hybridize with the targeting oligonucleotides to hybridize with the anchoring oligonucleotides (and thus generate a signal at the surface of the one or more substrates). In the presence of the target (when compared to the absence of the target), more signaling oligonucleotides will be available for hybridizing with the anchoring oligonucleotides and thus a greater signal will be generated at the surface of the one or more substrates. In the absence of the target (when compared to the presence of the target), less signaling oligonucleotides will be available for hybridizing with the anchoring oligonucleotides (because sequestered by the targeting oligonucleotides) and thus a lower signal will be generated at the surface of the one or more substrates. The sample can thus be characterized as having the target if it is determined that the test amount is higher than the control amount. The sample can thus be characterized as lacking the target if it is determined that the test amount is equal to or lower than the control amount.

In some embodiments in order to provide additional kinetic control to the method, it is possible to determine the dissociation constant (K_{D}) between the target and the targeting oligonucleotide. This step can be performed to select a targeting oligonucleotide having a K_{D} higher than the suspected concentration of the target in the sample and/or to dilute the sample prior to conducting the method. Alternatively or in combination, the method can also provide a step of providing a source of cations during the determination so as to increase k₂. Furthermore, the method can include applying a positive voltage to the one or more substrate so as to reduce k₃.

The method can rely on the use of one or more control systems. A positive control of the kits of the present disclosure can include, for example, a control signaling oligonucleotide capable of hybridizing to a control anchoring oligonucleotide and lacking a corresponding targeting oligonucleotide. The use of positive control provides a strong signal even in the absence of the target and thus allows determining if the generation of the signal is workable in the system. A negative control of the kits of the present disclosure can include, for example, a control signaling oligonucleotide capable of hybridizing to a control anchoring oligonucleotide and a (diffuse) control oligonucleotide complementary to the control signaling oligonucleotide. The use of the negative control provides a very low or no signal even in the presence of the target since the control signaling oligonucleotide will hybridize to the control oligonucleotide instead of the corresponding anchoring oligonucleotide and thus allows determining the background noise (if any) of the system.

The present invention will be more readily understood by referring to the following examples which are given to illustrate the invention rather than to limit its scope.

### Example

Materials.

Tris (2-carboxyethyl) phosphine hydrochloride (TCEP), 6-Mercaptohexanol (MCH), Guanidine hydrochloride and Quinine were purchased from Sigma Aldrich (St. Louis, Mo.). Thrombin was obtained from Cayman Chemical (Ann Arbor, Mich.). Polyclonal anti-digoxigenin was ordered from Roche Diagnostics (Indianapolis, Ind.). Whole blood (newborn calf) was purchased from Innovative research (Novi, Mich.). The regents and columns for DNA synthesis were obtained from Biosearch Technologies (Novato, Calif.) and ChemGenes Corporation (Wilmington, Mass.). The buffer used for quinine and antibody assay is 50 mM NaH₂PO₄, 150 mM NaCl, pH 7.0. The buffer used for thrombin assay is 50 mM Tris, 140 mM NaCl, 1 mM MgCl₂, pH 7.4.

DNA Sequence.

The nucleic acid sequences were synthesized using a DNA/RNA synthesizer (K&A Laborgeraete, Germany) and unlabeled DNAs were further purified by reverse-phase cartridge (RPC) while labeled DNAs were purified using high-performance liquid chromatography (HPLC) equipped with a XBridge Oligonucleotide BEH C18 column (130 Å, 2.5 μm, 4.6 mm×50 mm, 1/pkg). The HPLC purified oligonucleotide “signaling DNA-Dig”and “Anchoring DNA-Dig” were synthesized by Biosearch Technologies (Novato, Calif.). The sequences of DNAs are listed in Table 1.

TABLE 1
Sequences of aptamer, anchoring DNA, signaling DNA

| Notes | Sequence (5′-3′, SEQ ID NO:) |
|---|---|
| Quinine aptamer | GGG AGA CAA GGA AAA TCC TTC AAT GAA GTG GGT CGA CA |
| | (SEQ ID NO: 1) |
| Signaling DNA-12 | MB-GAA ATC CTT GTC TCC C (SEQ ID NO: 2) |
| Signaling DNA-14 | MB-GAT TTC CTT GTC TCC C (SEQ ID NO: 3) |
| Signaling DNA-16 | MB-ATT TTC CTT GTC TCC C (SEQ ID NO: 4) |
| Signaling DNA-18 | MB-GGA TTT TCC TTG TCT CCC (SEQ ID NO: 5) |
| Anchoring DNA-12 | GGG AGA CAA GGA TTT C-SH (SEQ ID NO: 6) |
| Anchoring DNA-14 | GGG AGA CAA GGA AAT C-SH (SEQ ID NO: 7) |
| Anchoring DNA-16 | GGG AGA CAA GGA AAA T-SH (SEQ ID NO: 8) |
| Anchoring DNA-18 | GAG ACA AGG AAA ATC C-SH (SEQ ID NO: 9) |
| Thrombin aptamer | AGT CCG TGG TAG GGC AGG TTG GGG TGA CT (SEQ ID NO: 10) |
| Signaling DNA-10thr | MB-CCA GAG ACC ACG GAC T (SEQ ID NO: 11) |
| Signaling DNA-12thr | MB-CCA GCT ACC ACG GAC T (SEQ ID NO: 12) |
| Signaling DNA-14thr | MB-CAC CCT ACC ACG GAC T (SEQ ID NO: 13) |
| Signaling DNA-16thr | MB-TGC CCT ACC ACG GAC T (SEQ ID NO: 14) |
| Anchoring DNA-10thr | AGT CCG TGG TCT CTG G-SH (SEQ ID NO: 15) |
| Anchoring DNA-12thr | AGT CCG TGG TAG CTG G-SH (SEQ ID NO: 16) |
| Anchoring DNA-14thr | AGT CCG TGG TAG GGT G-SH (SEQ ID NO: 17) |
| Anchoring DNA-16thr | AGT CCG TGG TAG GGC A-SH (SEQ ID NO: 18) |
| Signaling DNA-dig | Dig-CTT CTT CCC TTT CCT T-MB (SEQ ID NO: 19) |
| Anchoring DNA-dig | SH-AAG GAA AGG GAA GAA G (SEQ ID NO: 20) |
| Anchoring DNA-14d | 5′-G AGA CAA GGA AAA T-3′-SH (SEQ ID NO: 21) |
| Anchoring DNA-12d | 5′-GA CAA GGA AAA T-3′-SH (SEQ ID NO: 22). |

Gold Rod Electrode Functionalization and Electrochemical Measurement.

The gold working electrode (rod) (0.2 cm diameter, 0.0314 cm² surface area, West Lafayette, Ind.) were cleaned and functionalized as described in Xiao et al., 2007. Firstly, 1 μL of 100 μM Anchoring DNA was mixed with 2 μL of 10 mM TCEP for reduction of disulfide bonds at room temperature for 1 hour. Following, dilute the reduced Anchoring DNA solution to 100 nM and put the cleaned gold electrodes in the Anchoring DNA solution for 2-4 hours at room temperature (or overnight at 4° C.). The gold electrodes then were rinsed with deionized water and transferred into 2 mM MCH solution for removing physically adsorbed Anchoring DNA and passivating the rest surface of gold electrode. After incubation with MCH at room temperature for 2-4 hours, the gold electrodes were rinsed with deionized water again and can be stored in buffer at 4° C. before using. For the thrombin assay, the gold electrode was further treated with 0.1% BSA for 10 mins after MCH incubation to reduce non-specific adsorption of proteins on the gold electrode surface. The electrochemical measurements were initiated immediately after the addition of 100 nM signaling DNA and 100 nM aptamer into the sample containing the target molecules. The electrochemical measurements were conducted at room temperature using a EmStatMUX potentiostat multiplexer (Palmsens Instruments, Netherland) equipped with a standard three-electrodes cell containing a working electrode (gold rod electrode), a counter electrode (platinum, Sigma-Aldrich), and a reference electrode (Ag/AgCl (3 M NaCl), CH Instruments). The experimental data were recorded using square wave voltammetry in the range from −0.1 to −0.45 V in increments of 0.001 V vs. Ag/AgCl with an amplitude of 50 mV. The peak current was collected by using the manual fit mode in the PSTrace software of Palmsens Instruments, and the gain (%) represents the difference of peak current in the presence and absence of target molecules. The gold electrode with Anchoring DNA is readily regenerated by 6 M guanidine hydrochloride washing at room temperature.

Fluorescent Measurement.

Fluorescent experiments for the binding of FAM-Quinine aptamer-BHQ and signaling DNA-16u were performed using Cary Eclipse Fluorimeter at room temperature in the buffer of 50 mM NaH₂PO₄, 150 mM NaCl, pH 7.0. The fluorescent spectra were recorded at an excitation wavelength of 496 nm and an emission wavelength of 520 nm. The excitation and emission slits were set for 5.0 and 5.0 nm, respectively. Binding curves were obtained using 100 nM of Quinine aptamer-FB by sequentially increasing the concentration of signaling DNA-16u.

Fluorescent experiments for the binding of Quinine aptamer-FB and Quinine were obtained using an Applied Photophysics SX18.MV stopped-flow fluorimeter with by exciting at 480 (±5) nm and monitoring the total fluorescence above 495 nM using a cut-off filter. Binding curves were obtained using 100 nM of Quinine aptamer-FB by sequentially increasing the concentration of Quinine.

Kinetic Simulation.

The kinetic simulations were performed using the software MATLAB, and the language as follows:
k₁ and km₁ represent the association rate constant and dissociation rate constant of Ligand-Aptamer, respectively;
k₂ and km₂ represent the association rate constant and dissociation rate constant of Aptamer-Signaling DNA, respectively;
k₃ and km₃ represent the association rate constant and dissociation rate constant of Signaling DNA-Anchoring DNA, respectively;
% dQuinine/dt

dy(1)=−k_{1*}y(1)*y(2)+km1*y(5);
% dAptamer/dt

dy(2)=−k_{1*}y(1)*y(2)−k_{2*}y(2)*y(3)+km1*y(5)+km2*y(6);
% dSignaling DNA/dt

dy(3)=−k_{2*}y(2)*y(3)−k_{3*}y(3)*y(4)+km2*y(6)+km3*y(7);
% dAnchoring DNA/dt

dy(4)=−k_{3*}y(3)*y(4)+km3*y(7);
% d(Ligand-Aptamer)/dt

dy(5)=k_{1*}y(1)*y(2)−km1*y(5);
% d(Aptamer-Signaling DNA)/dt

dy(6)=k_{2*}y(2)*y(3)−km2*y(6);
% d(Signaling DNA-Anchoring DNA)/dt

dy(7)=k_{3*}y(3)*y(4)−km3*y(7);

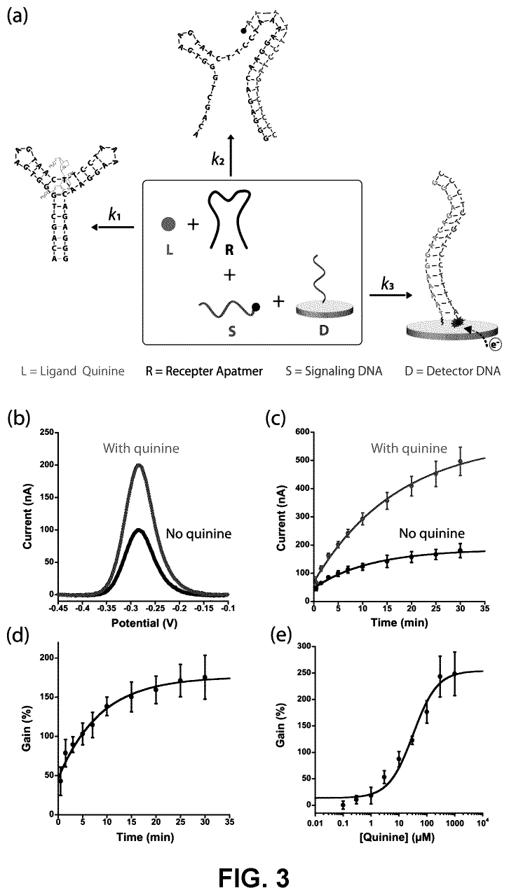

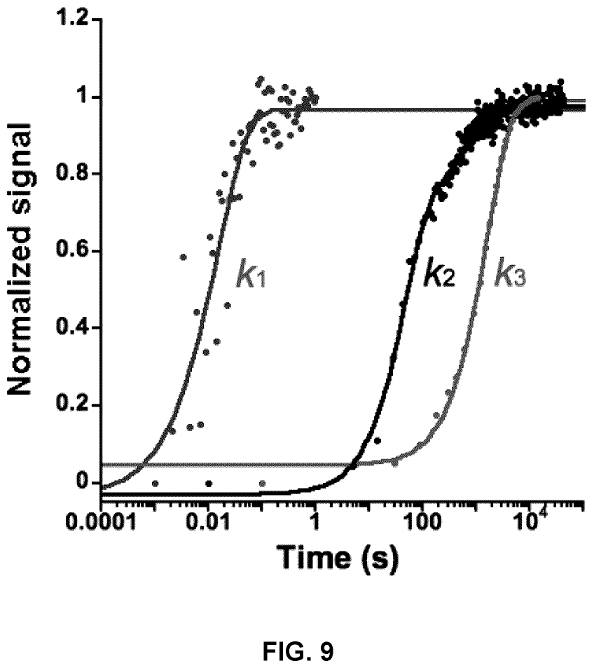

A one-pot DNA-based reactions was designed by employing a highly robust and selective DNA-based signaling mechanism that has been shown to be selective enough to work directly in whole blood. To do so, a 16 nucleotide redox-labeled “signaling” DNA and a complementary “anchoring” DNA strand attached to a gold electrode were used (see k₃, FIG. 3A). This reaction takes place in a minute time range (t_{1/2}=17.86 min, FIG. 9) and can be readily detected in undiluted whole blood samples (Mahshid et al., 2015). In order to render this signaling reaction sensitive to the presence of a specific ligand molecule, the signaling DNA was designed so that it is complementary to a specific receptor DNA aptamer (in this Example, a quinine binding aptamer was employed (Reinstein et al., 2013 and Porchetta et al., 2012) and the thrombin binding aptamers (Centi et al., 2007). If unbound to its ligand, this receptor aptamer acts like an inhibitor of the signaling mechanism by sequestrating the signaling strand thus preventing it to hybridize to the anchoring DNA on the electrode (see k₂, FIG. 3A). When the specific aptamer-binding target is present, this latter acts as an aptamer inhibitor by sequestrating it and preventing it to bind to the signaling DNA (see k₁, FIG. 3A). In such case, the signaling DNA is now free to hybridize to the anchoring DNA, creating a high electrochemical current in minutes through the formation of the Signaling-Anchoring complex. Interestingly, the rate of complex formation between the non-nucleic target and its aptamer receptor, k₁, is typically orders of magnitude faster than the rate of DNA-DNA hybridization in solution, k₂, due to charge repulsion between the DNA strands. Also, DNA hybridization rate on surface-bound DNA, k₃, is typically slower (FIG. 9).

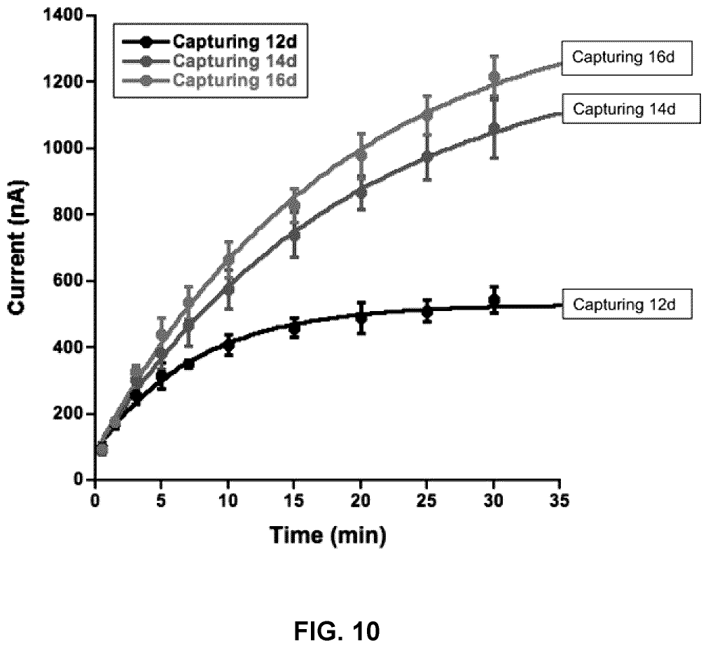

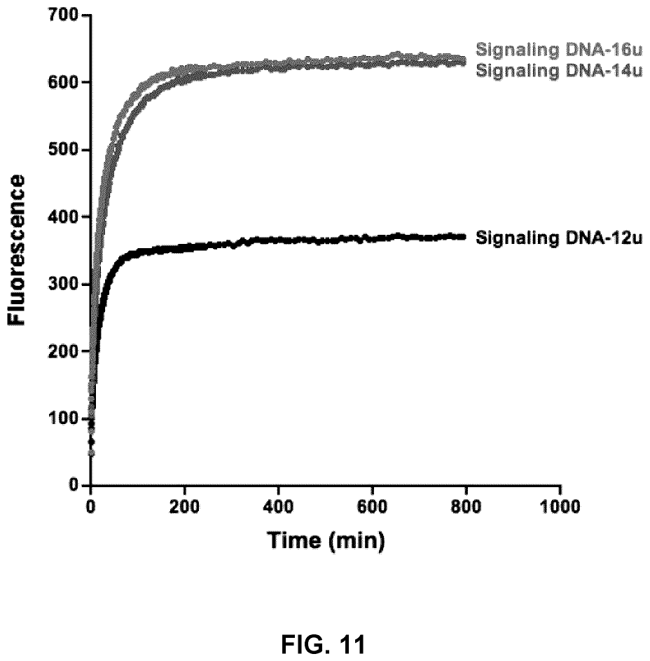

A signaling strand that forms 16 Watson Crick base-pair interaction both with the aptamer and anchoring strands was selected (FIG. 3A). This duplex length was found to enable efficient hybridization (>99%) to both the aptamer (Aptamer-Signaling) and the anchoring DNA (Signaling-anchoring) (FIGS. 10 and 11). A complementary 16-base DNA anchoring strand was attached to a gold electrode at high surface coverage using a 3′-C₆-thiol group that enables easy electrode attachment via the formation of sulfur-gold bond. The one-pot reaction was performed in a 1 mL volume using 100 nM of signaling DNA as this small concentration is enough to generate large p-amp current and only require small amount of reagents (e.g., 100 nM of aptamer DNA and between 5-100 nM of target).

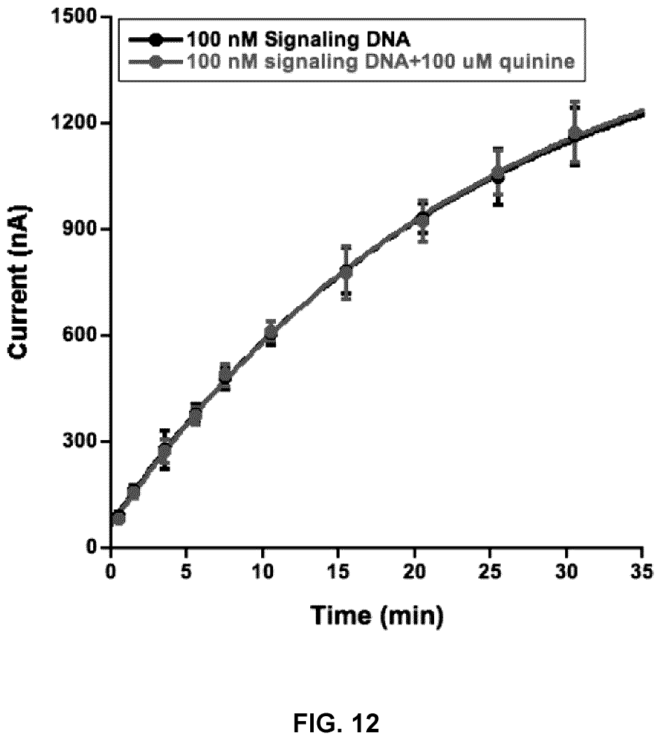

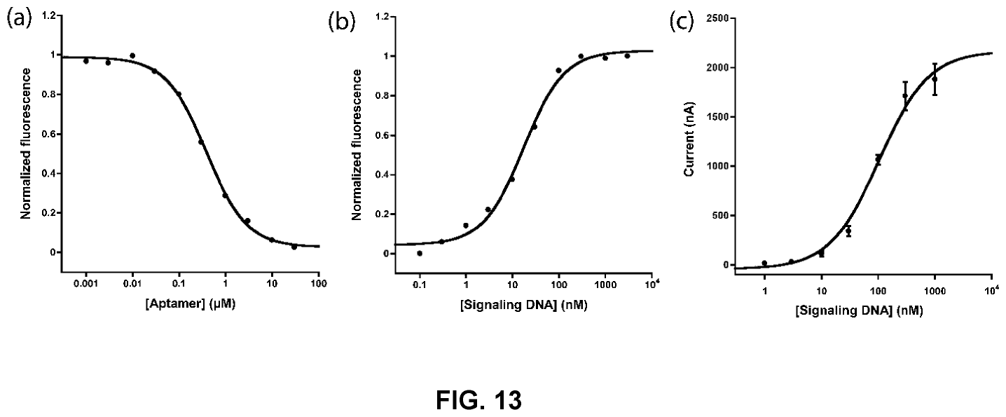

The first tested one-pot reaction produced a 100% increase in electrochemical current in the presence of 100 μM of quinine in the first 5 minutes of the reaction (FIGS. 3B-D). Two reactions were performed: in presence (100 μM) and absence of quinine. These reactions were triggered rapidly (<10 sec) adding 100 nM of aptamer and signaling DNA to solution containing (or not) quinine and by rapidly adding these solutions on the DNA functionalized electrode. After only 30 seconds the electrochemical current was already found 40% higher in the presence of quinine and kept increasing up to 175% after 30 min of reaction (FIG. 3D). An independent control in absence of quinine aptamer demonstrates that this increase in current is not only attributable to the presence of quinine (FIG. 12). It was found that the electrochemical current increases in a dose-dependent manner with a C50% of around 32.1 μM (±8.6) (FIG. 3E). When considering that the affinity (K_{d}) between the quinine and aptamer is around 0.39 μM (±0.03) (FIG. 13A), this suggests that there is a thermodynamic cost associated with the creation of this one-pot assay, which reduces the apparent affinity of the aptamer by ˜82.3-fold.

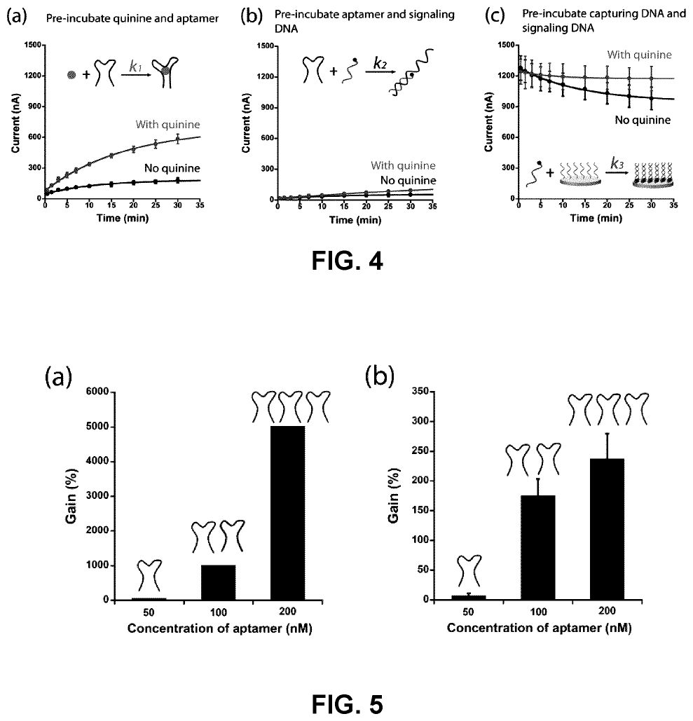

The performance of this one-pot assay was kinetically controlled. To demonstrate this, the same “one pot” three reactions was performed by preferentially favoring the formation of either the Target-Aptamer (k₁), Aptamer-Signaling (k₂), or Signaling-Anchoring (k₃) complexes (FIG. 4). For example, an increase in k₁ relative to k₂ and k₃ was mimicked by pre-incubating the target and aptamer (k) prior the addition of the signaling DNA and anchoring DNA reagents. Since k₁ is already much faster than k₂ and k₃ (at least 3 order of magnitudes, see FIG. 9), no significant difference in Signaling-Anchoring complex formation (electrochemical current) is detected (FIG. 4A). In contrast, the efficiency of the Signaling-Anchoring complex formation (i.e. electrochemical currents) drops dramatically (e.g., 84.1% reduction of signal in presence of quinine after 5 min) when triggering the Aptamer-Signaling reaction 30 minutes before adding Target and Anchoring DNA (i.e. when increasing k₂ to a point where it is faster than k₁) (FIG. 4B). These results suggest that once the Aptamer-Signaling complex forms, its dissociation rate (k₂) remains too slow to permit binding of quinine to the free Aptamer during the time course of this experience. Finally, a very high signal background (e.g., 11.8 fold higher after 5 minutes) was obtained in absence of quinine when triggering the Signaling-Anchoring reaction 30 minutes before adding the Target and Aptamer (i.e. when increasing k₃ to a point where it is faster than k₁ and k₂) (FIG. 4C). This later result suggests that once the Signaling-Anchoring complex forms, its dissociation rate (k₃, 0.069 min⁻¹) remains too slow (>30 min) to permit reversible binding of the signaling strand to the aptamer during the time course of this experiments. Overall these results demonstrate that the one-pot assay was kinetically controlled and does not reach equilibrium during the time frame of the experiment. These results also highlight the fact that the kinetics of all three reactions must be carefully programmed (k₁>k₂>k₃) in order for the one-pot reaction to achieve optimal gain rapidly.

In order to improve the gain and thus the performance of the one-pot assay, various experimental conditions were explored in order to change either k₂ or k₃. As shown in FIG. 2, as the difference between k₁, k₂, and k₃ increased, a drastic increase in the gain of the assay (smaller background and higher signal) was noticed. Since the rates of k₂ and k₃ were relatively close (see FIG. 9, within 15-fold), the conditions of the assay were modified in order to selectively increase k₂ or decrease k₃. In order to change k₂, the concentration of the aptamer was changed from 50 nM to 100 nM and 200 nM. Both the simulation and experimental results demonstrated an increase in overall gain when increasing k₂ relative to k₃ (FIG. 5). Similarly, the difference in rate between k₂ and k₃ could also be increased by slowing down k₃ (FIG. 6). To do so, the density of the anchoring strand (receptor) on the surface of the electrode was increased. This is known to slow down the kinetic of hybridization between an unbound and surface-attached DNA by increasing charge repulsion near the electrode surface (DNA is charges negatively). Both the simulation and experimental results demonstrated an increase in overall gain when decreasing k₃ relative to k₂ (FIG. 6).

The one-pot reaction system was very robust to large thermodynamic variations. For example, when the hybridization length was increased between the aptamer and signaling DNA from 12 to 18, therefore strongly favoring the formation of the Apt-Signaling complex, only a small variation in gain in observed (from 50% to 150%) (FIG. 7A).

The one-pot reaction strategy is likely universal and can be adapted for all aptamers (FIG. 7B). To demonstrate this, this assay was adapted for the detection of thrombin using the thrombin binding aptamer. The results shown in FIG. 7B indicate that similar gain and performance could be achieved with this assay without any complex design and optimization procedure.

Finally, as shown in FIG. 8B, the kinetically programmed one-pot system can be performed in a multiplex format directly in whole blood with other electrochemical DNA-based assays such as a steric-hindrance-based sensor (eSHHA) (Mahshid et al., 2015). For example, two electrodes, each functionalized with a specific anchoring DNA (i.e. a specific assay) can be used to detect quinine and antibody simultaneously in whole blood with outcross reactivity (FIGS. 8C and D)

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Centi, S.; Tombelli, S.; Minunni, M.; Mascini, M. Anal. Chem. 2007, 79, 1466.
Mahshid, S. S.; Camire, S.; Ricci, F.; Vallee-Belisle, A. J. Am. Chem. Soc. 2015, 137, 15596.
Porchetta, A.; Vallee-Belisle, A.; Plaxco, K. W.; Ricci, F. J. Am. Chem. Soc. 2012, 134, 20601.
Reinstein, O.; Yoo, M.; Han, C.; Palmo, T.; Beckham, S. A.; Wilce, M. C. J.; Johnson, P. E. Biochemistry 2013, 52, 8652.
Xiao, Y.; Lai, R.; Plaxco, K. W. Nat. Protoc. 2007, 2, 2875.

1. A system for detecting a target in sample, said system comprising:
a plurality of anchoring oligonucleotides each having a first end and a second free end;
a first substrate having a surface associated with, at a plurality of discrete locations, each of the first end of the plurality of anchoring oligonucleotides;
a plurality of signaling oligonucleotides, wherein each of the signaling oligonucleotide:
has a first nucleic acid sequence which is substantially complementary to a first region of each of the anchoring oligonucleotides and is capable of hybridizing with the anchoring oligonucleotides;
has a second nucleic acid sequence which is substantially complementary to a second region of each of a plurality of targeting oligonucleotides and is capable of hybridizing the targeting oligonucleotide;
wherein the first nucleic acid sequence and the second nucleic acid sequence are configured to avoid simultaneous hybridization of the signaling oligonucleotide with the anchoring oligonucleotide and the targeting oligonucleotide;
has a first end being associated with a reporter moiety and a second free end; and
is configured such that, upon hybridizing with the anchoring oligonucleotide, the first end of the signaling oligonucleotide is located in the vicinity of the first end of the anchoring oligonucleotide; and
the plurality of targeting oligonucleotides, wherein each of the targeting oligonucleotides:
is capable of specifically binding to the target;
has the second region which is substantially complementary to the second nucleic acid sequence of each of the signaling oligonucleotides and is capable of hybridizing with the signaling oligonucleotide; and
has a dissociation constant (K_{D}) with the target lower than the concentration of the target in the sample;
wherein the system is (i) kinetically controlled by a first association constant (k₁) between the targeting oligonucleotide and the target, a second association constant (k₂) between the targeting oligonucleotide and the signaling oligonucleotide and a third association constant (k₃) between the signaling oligonucleotide and an anchoring nucleotide and (ii) configured such that k₁>k₂>k₃.

2. The system of claim 1, wherein the system is configured such that k₁ is at least 10 times higher than k₂ and/or such that k₂ is at least 10 times higher than k₃.

3. (canceled)

4. The system of claim 1, wherein the molar concentration of the plurality of targeting oligonucleotides is equal to or higher than the molar concentration of the plurality of signaling oligonucleotides and/or the molar concentration of the plurality of the signaling oligonucleotides is equal to or higher than the amount of the plurality of anchoring oligonucleotides associated to the surface of the first substrate.

5. (canceled)

6. The system of claim 1, wherein the targeting oligonucleotide and/or the signaling oligonucleotide is configured so as to avoid hybridization between the targeting oligonucleotide and the signaling oligonucleotide in the presence of the target.

7. The system of claim 1, wherein the first end of each of the anchoring oligonucleotides is covalently associated to the surface of the first substrate.

8. The system of claim 1, wherein each of the anchoring oligonucleotide comprises at least 8 nucleic acid bases.

9. The system of, wherein the first region of the anchoring oligonucleotide is substantially identical to the second region of the targeting oligonucleotide.

10. The system of claim 1, wherein the first region of the anchoring oligonucleotide is complementary over the entire length of the first nucleic acid sequence of the signaling oligonucleotide.

11. The system of claim 1, wherein the first substrate is a metallic electrode.

12. (canceled)

13. The system of claim 1, wherein the first nucleic acid sequence of the signaling oligonucleotide is complementary over the entire length of the first region of the anchoring oligonucleotide.

14. The system of claim 1, wherein the second nucleic acid sequence of the signaling oligonucleotide is complementary over the entire length of the second region of the targeting oligonucleotide.

15. The system of claim 1, wherein the first nucleic acid sequence is the second nucleic acid sequence.

16. The system of claim 1, wherein each of the signaling oligonucleotide comprises at least 8 nucleic acid bases.

17. The system of claim 1, wherein the reporter moiety is a redox-reporter.

18. (canceled)

19. The system of claim 1, wherein the targeting oligonucleotide is an aptamer.

20. The system of claim 1, wherein the second region of each of the targeting oligonucleotides is substantially identical to the first region of the anchoring oligonucleotide.

21. The system of claim 1, wherein the second region of each of the targeting oligonucleotide is complementary over the entire length to the second nucleic acid sequence of the signaling oligonucleotide.

22. The system of claim 1, wherein the targeting oligonucleotide is at least 10 nucleic acid bases.

23. The system of claim 1 for detecting a plurality of distinct targets, said system further comprising:
a plurality of types of anchoring oligonucleotides each anchoring oligonucleotide type having a distinct nucleic acid sequence, a first end and a second free end;
a plurality of types of substrates, each of the substrate type having a surface associated with, at a plurality of discrete locations, with the first end of a single type of anchoring oligonucleotides and each of the substrates having a different type of anchoring oligonucleotide;
a plurality of types of signaling oligonucleotides, wherein each type of the signaling oligonucleotides:
has a first nucleic acid sequence which is substantially complementary to a first region of a corresponding anchoring oligonucleotide type and is capable of hybridizing with the corresponding anchoring oligonucleotide type;
has a second nucleic acid sequence which is substantially complementary to a second region of each of a plurality of targeting oligonucleotide type and is capable of hybridizing with the targeting oligonucleotide;
wherein the first nucleic acid sequence and the second nucleic acid sequence are configured to avoid the simultaneous hybridization of the signaling oligonucleotide with the anchoring oligonucleotide and the targeting oligonucleotide;
has a first end being associated with a reporter moiety; and
is configured such that, upon hybridizing with the corresponding anchoring oligonucleotide, the second end of the signaling oligonucleotide is located in the vicinity of the first end of the anchoring oligonucleotide; and
the plurality of types of targeting oligonucleotides wherein each type targeting oligonucleotides:
is capable of specifically binding to a corresponding target;
has the second region which is substantially complementary to the second nucleic acid sequence of each of the signaling oligonucleotides and is capable of hybridizing with the signaling oligonucleotide; and
has a dissociation constant (K_{D}) with the target lower than the concentration of the target in the sample.

24. A method for the detection of a target in a sample, said method comprising:
(a) providing the sample suspected of having the target and the system of claim 1 comprising a plurality of anchoring oligonucleotides, a first substrate, a plurality of signaling oligonucleotides and a plurality of targeting oligonucleotides;
(b) providing or determining a control amount of the plurality of anchoring oligonucleotide having hybridized with the signaling oligonucleotide in the absence of the target;
(c) contacting the sample with the plurality of signaling oligonucleotides and the plurality of targeting oligonucleotides in the absence of the anchoring oligonucleotides to provide a targeted mixture;
(d) contacting the targeted mixture with the plurality of anchoring oligonucleotides associated with the first substrate to provide a detectable mixture;
(e) determining a test amount of the plurality of anchoring oligonucleotides having hybridized with the plurality signaling oligonucleotides in the system in the presence of the detectable mixture; and
(f) characterizing the sample has having the target if it is determined that the test amount is higher than the control amount and as lacking the target if it is determined that the test amount is equal to or lower than the control amount;
wherein the method is (i) kinetically controlled by a first association constant (k₁) between the targeting oligonucleotide and the target, a second association constant (k₂) between the targeting oligonucleotide and the signaling oligonucleotide and a third association constant (k₃) between the signaling oligonucleotide and an anchoring nucleotide and (ii) in which k₁>k₂>k₃.

25-36. (canceled)


SEQUENCE LISTING (22 sequences)

SEQ ID NO: 1 - DNA, 38 bp, Artificial Sequence
  Quinine aptamer
gggagacaaggaaaatccttcaatgaagtgggtcgaca

SEQ ID NO: 2 - DNA, 16 bp, Artificial Sequence
  Signaling DNA-12
gaaatccttgtctccc

SEQ ID NO: 3 - DNA, 16 bp, Artificial Sequence
  Signaling DNA-14
gatttccttgtctccc

SEQ ID NO: 4 - DNA, 16 bp, Artificial Sequence
  Signaling DNA-16
attttccttgtctccc

SEQ ID NO: 5 - DNA, 18 bp, Artificial Sequence
  Signaling DNA-18
ggattttccttgtctccc

SEQ ID NO: 6 - DNA, 16 bp, Artificial Sequence
  Anchoring DNA-12
gggagacaaggatttc

SEQ ID NO: 7 - DNA, 16 bp, Artificial Sequence
  Anchoring DNA-14
gggagacaaggaaatc

SEQ ID NO: 8 - DNA, 16 bp, Artificial Sequence
  Anchoring DNA-16
gggagacaaggaaaat

SEQ ID NO: 9 - DNA, 16 bp, Artificial Sequence
  Anchoring DNA-18
gagacaaggaaaatcc

SEQ ID NO: 10 - DNA, 29 bp, Artificial Sequence
  Thrombin aptamer
agtccgtggtagggcaggttggggtgact

SEQ ID NO: 11 - DNA, 16 bp, Artificial Sequence
  Signaling DNA-10thr
ccagagaccacggact

SEQ ID NO: 12 - DNA, 16 bp, Artificial Sequence
  Signaling DNA-12thr
ccagctaccacggact

SEQ ID NO: 13 - DNA, 16 bp, Artificial Sequence
  Signaling DNA-14thr
caccctaccacggact

SEQ ID NO: 14 - DNA, 16 bp, Artificial Sequence
  Signaling DNA-16thr
tgccctaccacggact

SEQ ID NO: 15 - DNA, 16 bp, Artificial Sequence
  Anchoring DNA-10thr
agtccgtggtctctgg

SEQ ID NO: 16 - DNA, 16 bp, Artificial Sequence
  Anchoring DNA-12thr
agtccgtggtagctgg

SEQ ID NO: 17 - DNA, 16 bp, Artificial Sequence
  Anchoring DNA-14thr
agtccgtggtagggtg

SEQ ID NO: 18 - DNA, 16 bp, Artificial Sequence
  Anchoring DNA-16thr
agtccgtggtagggca

SEQ ID NO: 19 - DNA, 16 bp, Artificial Sequence
  Signaling DNA-dig
cttcttccctttcctt

SEQ ID NO: 20 - DNA, 16 bp, Artificial Sequence
  Anchoring DNA-dig
aaggaaagggaagaag

SEQ ID NO: 21 - DNA, 14 bp, Artificial Sequence
  Anchoring DNA-14d
gagacaaggaaaat

SEQ ID NO: 22 - DNA, 12 bp, Artificial Sequence
  Anchoring DNA-12d
gacaaggaaaat